(19) United States
(12) Patent Application Publication
(10) Pub. No.: US 2023/0075765 A1
(43) Pub. Date: Mar. 9, 2023

(54) PYRROLO[2,3-D]PYRIMIDINE DERIVATIVE TARGETING EGFR MUTATION, AS WELL AS THE PREPARATIVE METHOD AND THE USE THEREOF

(71) Applicants: WEST CHINA HOSPITAL, SICHUAN UNIVERSITY, Chengdu, Sichuan (CN); CHENGDU WEST CHINA PRECISION MEDICINE INDUSTRY TECHNOLOGY RESEARCH INSTITUTE CO., LTD., Chengdu, Sichuan (CN)

(72) Inventors: Weimin LI, Chengdu, Sichuan (CN); Yang HE, Chengdu, Sichuan (CN); Zhenqiang XIA, Chengdu, Sichuan (CN); Ridong HUANG, Chengdu, Sichuan (CN); Hai CHEN, Chengdu, Sichuan (CN); Xinglong ZHOU, Chengdu, Sichuan (CN); Yingying CHAI, Chengdu, Sichuan (CN)

(21) Appl. No.: 17/758,344

(22) PCT Filed: Nov. 10, 2020
(86) PCT No.: PCT/CN2020/127884

§ 371 Date: Jul. 1, 2022

(30) Foreign Application Priority Data
Aug. 28, 2020 (CN) 202010888223.1

(51) Int. Cl. C07D 487/04; A61P 35/00; A61P 35/02

(52) U.S. Cl. CPC C07D 487/04; A61P 35/00; A61P 35/02

(57) ABSTRACT

A pyrrolo[2,3-d]pyrimidine derivative targeting EGFR mutations, as well as the preparative method and the use thereof are provided. The derivative is a compound of formula I, or a salt thereof, or a stereoisomer thereof. It has low toxicity to normal cells and has a significant inhibitory effect on lung cancer cell lines, especially has good selectivity and significant inhibitory effect for EGFR-mutant HCC827 cells, against the phosphorylation of EGFR, and against mutant EGFR. It can be used to treat lung cancer, especially non-small cell lung cancer and can also be used to prepare tyrosine kinase inhibitors, especially the inhibitors of EGFR phosphorylation.

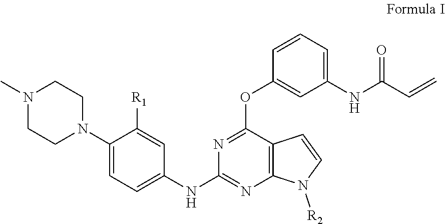

### TECHNICAL FIELD

The present invention belongs to the field of chemistry and medicine, and specifically relates to a pyrrolo[2,3-d]pyrimidine derivative targeting EGFR mutation, as well as the preparative method and the use thereof.

### BACKGROUND TECHNOLOGY

Lung cancer is the malignant tumor with the highest morbidity and mortality in the world. The number of deaths caused by lung cancer reaches 1.6 million each year. It is the main reason of cancer-related deaths in men (22% of cancer deaths) and women (13.8% of cancer deaths). In the United States, approximate 14% of new cancers are lung cancer. The American Cancer Society estimated that there were approximately 234,000 lung cancer cases in the United States in 2018, and approximately 154,000 deaths. The situation in our country is equally serious. According to China's latest cancer data released by the National Cancer Center in January 2019, lung cancer was the most common malignant tumor in my country in terms of morbidity and mortality, which posed a great threat to national health and brings great pressure for the social and economic development. Non-small cell lung cancer accounts for about 85% of all lung cancers, and these patients have the same histological subtype. Non-small cell lung cancer includes lung adenocarcinoma (LUAD), lung squamous cell carcinoma (LUSC) and large cell carcinoma.

In the past 20 years, many advances have been made in the treatment of lung cancer, including the development of methods for specific molecular subtypes of lung adenocarcinoma as well as the development of new methods for the treatment of lung squamous cell carcinoma. Currently, the drug treatment methods for lung cancer mainly include chemotherapy, immunotherapy, and molecular targeted therapy.

EGFR is a member of the tyrosine kinase type I receptor family, and its gene is located on human chromosome 7. There are 28 exons in EGFR, which can form a protein distributed on the cell membrane of various epithelial cells, bind with epidermal growth factor or EGF, and regulate cell growth. In contrast, in EGFR mutations of non-small cell lung cancer, the insertions of exon 20 and the mutations of exon 18 are less common than the deletions of exon 19 and the substitutions of 21 L858R exon. The activation and regulation of EGFR and its downstream genes lead to cell proliferation, apoptosis, and angiogenesis. Some drugs for EGFR mutations have been developed, such as tyrosine kinase inhibitors (TKIs) and BRAF inhibitors.

In the late 1990s, with the advent of the oral EGFR tyrosine kinase inhibitor (TKI) gefitinib, molecular targeted therapy for NSCLC patients was applied for the first time. Erlotinib is another TKI that targets EGFR. For patients with advanced non-small cell lung cancer, this drug leads to a higher survival rate than maintenance therapy. Studies have shown that most patients who benefit from EGFR TKIs have been observed with the activation of EGFR mutations. Additional genetic changes including ALK rearrangement, ROS1 fusion and BRAF mutation have effectively promoted the development of targeted therapy.

In the past few decades, tyrosine kinase inhibitors have been regarded as effective drugs for the treatment of non-small cell lung cancer, as well as good targeted drugs. There are many drugs targeting EGFR, such as gefitinib, erlotinib, cetuximab and panitumumab. Some studies have shown that, compared with the first-line chemotherapy, two first-generation EGFR TKIs (gefitinib and erlotinib) have substantial benefits in PFS. However, after chemotherapy in patients with advanced NSCLC, the effect of EGFR-TKI on OS is not obvious. Some studies have shown that patients develop resistance after subjecting to the first-generation EGFR-TKI treatment for 10 to 14 months. The resistance mechanism of the first generation of EGFR-TKI in NSCLC has been identified as TK domain mutation (T790M), MET amplification, RAS mutation and so on. Mutations in the TK region (T790M) are considered to be the most common acquired resistance mutations in NSCLC patients. Some NSCLC patients with T790M mutations have never received EGFR-TKI treatment. These findings suggest that the T790M mutation is a potential target for NSCLC patients. Therefore, new measures and therapies need to be developed to overcome drug resistance.

In recent years, osimertinib has emerged as the third-generation EGFR-TKIs, which can target sensitive and drug-resistant EGFR mutations (T790M). In the FLAURA study, the median PFS (18.9 months) of osimertinib-treated NSCLC patients was significantly longer than that of patients using first-generation EGFR-TKIs (gefitinib and erlotinib) (10.2 months). Unfortunately, a study has shown that osimertinib resistance has emerged. EGFR C797S mutations, PIK3CA mutations, KRAS mutations, BRAF mutations, and MET amplifications have appeared in 45 patients. In NSCLC with EGFR mutations, 5-20% of EGFR-TKI drug-resistant patients have MET amplification. MET amplification increased the proliferation and the migration of HCC827 cells in NSCLC. Therefore, there is still a need to continuously develop and update the third-generation of EGFR-TKI targeted drug molecules.

### CONTENT OF THE INVENTION

The object of the present invention is to provide a pyrrolo[2,3-d]pyrimidine derivative targeting EGFR mutation, as well as a preparative method and a use thereof.

The present invention provides a compound of formula I, or a salt thereof, or a stereoisomer thereof:

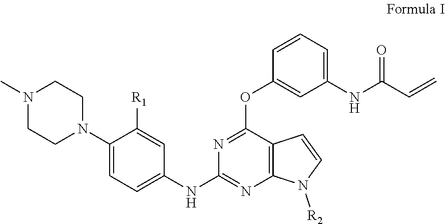

wherein,
R₁ is selected from the group consisting of halogen, C₁˜C₈ alkyl, C₁˜C₈ alkoxy, hydroxyl, nitro, amino or carboxyl; but R₁ is not a fluorine;
R₂ is selected from the group consisting of H, halogen, C₁˜C₈ alkyl, C₁˜C₈ alkoxy, hydroxyl, nitro, amino, carboxyl or —(CH₂)ₙ—O—C(O)—R₃;
n is selected from an integer from 1 to 8;
R₃ is selected from H or C₁˜C₈ alkyl.

Further,

R₁ is selected from the group consisting of chlorine, bromine, iodine, C₁˜C₄ alkyl, C₁˜C₄ alkoxy, hydroxyl, nitro, amino or carboxyl;
R₂ is selected from the group consisting of H, halogen, C₁˜C₄ alkyl, C₁˜C₄ alkoxy, hydroxyl, nitro, amino, carboxyl or —(CH₂)ₙ—O—C(O)—R₃;
n is selected from an integer from 1 to 4;
R₃ is selected from H or C₁˜C₄ alkyl.

Preferably,

R₃ is selected from H or t-butyl.

Further, said compound has the structure of formula II:

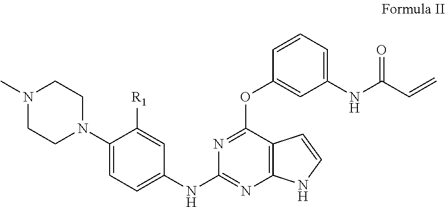

wherein,
R₁ is selected from the group consisting of halogen, C₁˜C₈ alkyl, C₁˜C₈ alkoxy, hydroxyl, nitro, amino or carboxyl; but R₁ is not a fluorine;

Preferably, R₁ is selected from the group consisting of chlorine, bromine, iodine, C₁˜C₄ alkyl, C₁˜C₄ alkoxy, hydroxyl, nitro, amino or carboxyl; More preferably, R₁ is selected from the group consisting of chlorine, bromine, iodine, C₁˜C₃ alkyl, C₁˜C₃ alkoxy, nitro or amino.

Further, said compound has the structure of formula III:

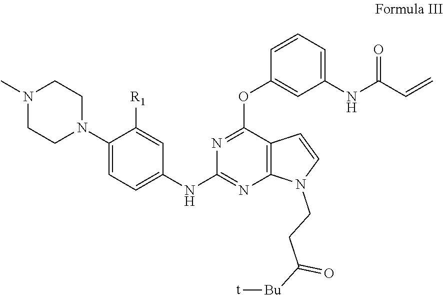

wherein,
R₁ is selected from the group consisting of halogen, C₁˜C₈ alkyl, C₁˜C₈ alkoxy, hydroxyl, nitro, amino or carboxyl; but R₁ is not a fluorine;

Preferably, R₁ is selected from the group consisting of chlorine, bromine, iodine, C₁˜C₄ alkyl, C₁˜C₄ alkoxy, hydroxyl, nitro, amino or carboxyl;

More preferably, R₁ is selected from the group consisting of chlorine, bromine, iodine, C₁˜C₃ alkyl, C₁˜C₃ alkoxy, nitro or amino.

Further, said compound is one of the following compounds:

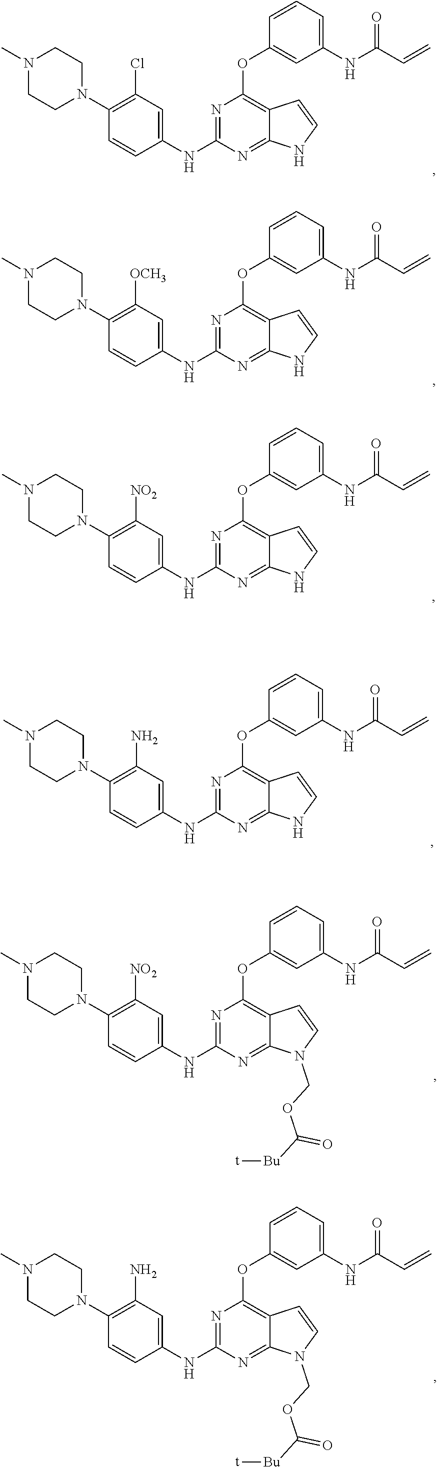

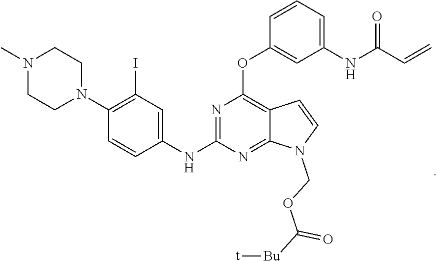

The present invention provides the use of the compound mentioned above, or a salt thereof, or a stereoisomer thereof in the preparation of a tyrosine kinase inhibitor.

Further, said tyrosine kinase inhibitor is a drug that inhibits the phosphorylation of EGFR.

Further, said tyrosine kinase inhibitor is a drug for the treatment of cancers; Preferably, the cancer is lung cancer, liver cancer, gastric cancer, kidney cancer, breast cancer, esophageal cancer, nasopharyngeal cancer, uterine cancer, colon cancer, rectal cancer, leukemia, bone cancer, and lymphoma.

Further, the cancer is lung cancer; preferably, the lung cancer is non-small cell lung cancer; more preferably, the lung cancer is EGFR-mutant non-small cell lung cancer.

The present invention also provides a drug, which is a preparation prepared from the compound mentioned above, or a salt thereof, or a stereoisomer thereof as an active ingredient, with the addition of pharmaceutically acceptable excipients or auxiliary ingredients.

The compounds and derivatives provided in the present invention can be named according to IUPAC (International Union of Pure and Applied Chemistry) or CAS (Chemical Abstracting Service, Columbus, Ohio) naming system.

For the definition of the term used in the present invention: unless otherwise specified, the initial definition provided for the group or the term herein is applicable to those in the whole specification; for terms not specifically defined herein, according to the disclosure content and the context, the term should have the meaning commonly given by those skilled in the field.

In the present invention, halogen is fluorine, chlorine, bromine, or iodine.

“Alkyl” is a hydrocarbon group formed by losing one hydrogen in an alkane molecule, such as methyl —CH₃, ethyl —CH₃CH₂, etc. “C₁₋₈ alkyls” denotes a straight or branched hydrocarbon chain containing 1-8 carbon atoms.

“C₁-C₈ alkoxy” denotes an alkoxy containing one to eight carbon atoms.

In the present invention, room temperature is 25±5° C.; overnight is 12±2 h.

The compound of the present invention has low toxicity to normal cells, but has obvious inhibitory effect on lung cancer cell lines, and especially has good selectivity and significant inhibitory effect for EGFR mutant HCC827 cells. The compound of the present invention can induce the apoptosis of EGFR mutant cells H1975 cells and HCC827 cells, and at the same time block the cycle of both cells in the G0/G1 phase. Meanwhile, the compound of the present invention can effectively inhibit the phosphorylation of EGFR, as well as the phosphorylation of two important kinases Akt and ERK1/2 in the downstream of the pathway involved in the proliferation and survival of cancer cells in H1975 cells. The compound of the present invention can be used to treat lung cancer, especially non-small cell lung cancer, which has a strong inhibitory effect on EGFR-mutant lung cancer and is less toxic. The present invention can also be used to prepare tyrosine kinase inhibitors, especially the inhibitors of EGFR phosphorylation, and have good application prospects.

Obviously, based on above content of the present invention, according to the common technical knowledge and the conventional means in the field, without department from above basic technical spirits, other various modifications, alternations, or changes can further be made.

By following specific examples of said embodiments, the above content of the present invention is further illustrated. But it should not be construed that the scope of above subject of the present invention is limited to following examples. The techniques realized based on above content of the present invention are all within the scope of the present invention.

### DESCRIPTION OF FIGURES

FIG. 1. ¹H NMR spectrum of intermediate compound 19.

FIG. 2. ¹H NMR spectrum of intermediate compound 20.

FIG. 3. ¹H NMR spectrum of compound 1 according to the present invention.

FIG. 4. ¹H NMR spectrum of compound 2 according to the present invention.

FIG. 5. ¹H NMR spectrum of compound 3 according to the present invention.

FIG. 6. ¹H NMR spectrum of compound 4 according to the present invention.

FIG. 7. ¹H NMR spectrum of compound 5 according to the present invention.

FIG. 8. ¹H NMR spectrum of compound 6 according to the present invention.

FIG. 9. ¹H NMR spectrum of compound 7 according to the present invention.

FIG. 10. ¹H NMR spectrum of compound 8 according to the present invention.

FIG. 11. ¹H NMR spectrum of compound 9 according to the present invention.

FIG. 12. The inhibitory activity (IC₅₀/μmol) of different compounds according to the present invention on normal cells and different lung cancer cell lines.

FIG. 13. The inhibitory effect of compound 5 on phosphorylation of EGFR and related proteins in different cells.

FIG. 14. The inhibitory effect of compound 6 on phosphorylation of EGFR and related proteins in different cells.

FIG. 15. The inhibitory effect of avitinib on phosphorylation of EGFR and related proteins in different cells.

FIG. 16. The effect of compound 5, compound 6 and avitinib on the apoptosis of HCC827 cells.

FIG. 17. The effect of compound 5, compound 6 and avitinib on the apoptosis of H1975 cells.

FIG. 18. The effect of compound 5, compound 6 and avitinib on the apoptosis of HCC827 cells.

FIG. 19. The effect of compound 5, compound 6 and avitinib on the cycle of H1975 cells.

### EXAMPLES

Unless otherwise stated, the starting materials and equipment used in the specific examples of the present invention are all known products and can be obtained by purchasing commercially available items. The main reagents are shown in Table 1.

TABLE 1
Some experimental reagents of the present invention.

| Reagents | Manufacturer | Purity |
|---|---|---|
| 2,6-dichloro-7-deazapurine | Sinopharm Group | 98% |
| chloromethyl pivalate | Sinopharm Group | 99% |
| 3 -nitrophenol | Sinopharm Group | 99% |
| 3-fluoro-4-(4-methyl- | Sinopharm Group | 97% |
| piperazinyl)aniline | | |
| 2-iodo-4-nitrofluorobenzene | Sinopharm Group | 97% |
| reduced iron powder | Sinopharm Group | 98% |
| ammonium chloride | Kelong Chemical | 99% |
| | Engineering | |
| | Factory | |
| Palladium on carbon | Sinopharm Group | >90% |
| hydrazine hydrate | Sinopharm Group | >98% |
| isopropanol | Kelong Chemical | HPLC |
| | Engineering | |
| | Factory | |
| t-butanol | Kelong Chemical | AR |
| | Engineering | |
| | Factory | |
| anhydrous sodium sulfate | Kelong Chemical | 99% |
| | Engineering | |
| | Factory | |
| 4-fluoro-3-nitroaniline | Sinopharm Group | 98% |
| 4-(4-methyl-1-piperazinyl)aniline | Sinopharm Group | 98% |
| 2-fluoro-5-nitroanisole | Sinopharm Group | 97% |
| 1-(4-amino-2-chlorophenyl)-4- | Sinopharm Group | 98% |
| methylpiperazine | | |
| 2-fluoro-5-nitrophenol | Sinopharm Group | 98% |
| acryloyl chloride | Sinopharm Group | 98% |
| XPhOs | Sinopharm Group | 97% |
| tris(dibenzylidene- | Sinopharm Group | 97% |
| acetone)dipalladium | | |
| SnCl | Sinopharm Group | 98% |
| NaI | Sinopharm Group | 98% |
| AgF | Sinopharm Group | 98% |
| CuO | Sinopharm Group | 98% |
| K₂CO₃ | Kelong Chemical | AR |
| | Engineering | |
| | Factory | |
| ethanol | Kelong Chemical | HPLC |
| | Engineering | |
| | Factory | |
| acetonitrile | Kelong Chemical | HPLC |
| | Engineering | |
| | Factory | |
| 1,4-dioxane | Kelong Chemical | AR |
| | Engineering | |
| | Factory | |
| DMSO-d₆ | Sinopharm Group | 99.9% |
| dichloromethane-d₂ | Sinopharm Group | 99.99% |

Main instrument:
(1) Mass spectrometer: Q-TOF spectrometer, ESI ionization source, Bruker, Germany;
(2) Nuclear magnetic resonance instrument: AV II-400 MHz, AV II-600 MHz or AV II-800 MHz, with TMS an internal standard, Bruker, Germany.

### Example 1 Synthesis of Key Intermediate 1

The synthetic route of key intermediate 1 was as follows:

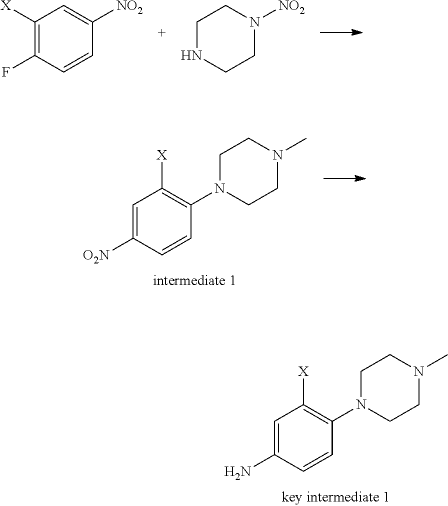

wherein,
when X=Br, intermediate 1 is compound 10, and key intermediate 1 is compound 14;
when X=I, intermediate 1 is compound 11, and key intermediate 1 is compound 15;
when X=OH, intermediate 1 is compound 12, and key intermediate 1 is compound 16;
when X=OCH₃, intermediate 1 is compound 13, and key intermediate 1 is compound 17.
or, the synthetic route of key intermediate 1 is as follows:

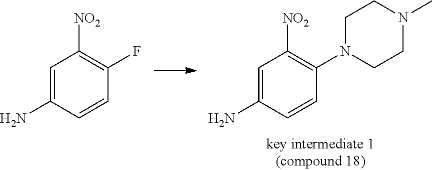 [SMILES: Nc1ccc(F)c([N+](=O)[O-])c1 ; CN1CCN(c2ccc(N)cc2[N+](=O)[O-])CC1]

when X=NO₂, key intermediate 1 is compound 18.

The specific preparative method was as follows:

(1) Synthesis of 1-(2-iodo-4-nitrophenyl)-4-methylpiperazine (11): the mixed solution of 4-fluoro-3-iodonitrobenzene (1.34 g, 5.0 mmol) and N-methylpiperazine (about 6 ml, 52 mmol) was heated to 90° C. and reacted for more than 5 h. The reaction solution was naturally cooled to room temperature, to which was added water to dilute, and then the precipitation deposited. The resultant solid was filtered by suction, washed with water, and dried to obtain 1.67 g of the product (compound 11), with a yield of 96%. HRMS (ESI⁺) m/z: calcd for C₁₁H₁₄IN₃O₂: 348.0131 [M+H]⁺; Found 348.0204 [M+H]⁺.

(2) Synthesis of 3-iodo-4-(4-methylpiperazine)-aniline (15): compound 11 (1.39 g, 4 mmol) and Pd/C (0.3 g) were dissolved in 50 ml isopropanol, mixed, and heated under reflux. Hydrazine hydrate (2 ml, dissolved in 10 ml isopropanol) was added dropwise to the reflux solution, and the mixture was allowed to react for 1 h. The reaction solution was filtered after cooling, and the filtrate was concentrated under reduced pressure to provide 1.23 g of the product (compound 15), with a yield of 97%. HRMS-ESI (m/z): calcd for C₁₁H₁₆IN₃: 318.0389 [M+H]⁺; Found 318.0468 [M+H]⁺. ¹H NMR (400 MHz, Chloroform-d) δ 7.23-7.18 (m, 1H), 6.89 (d, J=8.4 Hz, 1H), 6.69-6.59 (m, 1H), 3.48 (d, J=37.4 Hz, 2H), 2.93 (d, J=5.0 Hz, 4H), 2.73-2.57 (m, 4H), 2.39 (s, 3H).

(3) Synthesis of 1-(2-bromo-4-nitrophenyl)-4-methylpiperazine (10): the mixed solution of 4-fluoro-3-bromonitrobenzene (1.09 g, 5.0 mmol) and N-methylpiperazine (about 6 ml, 52 mmol) was heated to 90° C. and reacted for more than 5 h. The reaction solution was naturally cooled to room temperature, to which was added water to dilute, and then the precipitation deposited. The resultant solid was filtered by suction, washed with water, and dried to obtain 1.42 g of the product (compound 10), with a yield of 95%. HRMS (ESI⁺) m/z: calcd for C₁₁H₁₄BrN₃O₂: 300.0269 [M+H]⁺, 322.0269 [M+Na]⁺; Found 300.0348 [M+H]⁺, 322.0165 [M+Na]⁺.

(4) Synthesis of 3-bromo-4-(4-methylpiperazine)-aniline (14): compound 10 (1.22 g, 4.08 mmol) and Pd/C (0.3 g) were dissolved in 50 ml isopropanol, mixed, and heated under reflux. Hydrazine hydrate (2 ml, dissolved in 10 ml isopropanol) was added dropwise to the reflux solution, and the mixture was allowed to react for 1 h. The reaction solution was filtered after cooling, and the filtrate was concentrated under reduced pressure to provide 1.05 g of the product (compound 14), with a yield of 96%. HRMS-ESI (m z) calcd for C₁₁H₁₆BrN₃: 270.0528 [M+H]⁺; Found 270.0600 [M+H]⁺.

(5) Synthesis of 1-(2-methoxy-4-nitrophenyl)-4-methylpiperazine (13): the mixed solution of 1-fluoro-2-methoxy-4-nitrobenzene (0.85 g, 5.0 mmol) and N-methylpiperazine (about 6 ml, 52 mmol) was heated to 90° C. and reacted for more than 5 h. The reaction solution was naturally cooled to room temperature, to which was added water to dilute, and then the precipitation deposited. The resultant solid was filtered by suction, washed with water, and dried to obtain 1.17 g of the product (compound 13), with a yield of 93%. HRMS-ESI (m z) calcd for C₁₂H₁₇N₃O₃: 252.1270 [M+H]⁺; Found 252.1341 [M+H]⁺.

(6) Synthesis of 3-methoxy-4-(4-methylpiperazine)-aniline (17): compound 13 (1.22 g, 4.86 mmol) and Pd/C (0.3 g) were dissolved in 50 ml isopropanol, mixed, and heated under reflux. Hydrazine hydrate (2 ml, dissolved in 10 ml isopropanol) was added dropwise to the reflux solution, and the mixture was allowed to react for 1 h. The reaction solution was filtered after cooling, and the filtrate was concentrated under reduced pressure to provide 1.03 g of the product (compound 17), with a yield of 96%. HRMS-ESI (m z) calcd for C₁₂H₁₉N₃O: 222.1528 [M+H]⁺; Found 222.1603 [M+H]⁺. ¹H NMR (400 MHz, Chloroform-d) δ 6.78 (d, J=8.0 Hz, 1H), 6.25 (d, J=8.5 Hz, 2H), 3.80 (s, 3H), 3.49 (s, 2H), 3.01 (s, 4H), 2.63 (s, 4H), 2.36 (s, 3H).

(7) Synthesis of 2-(4-methylpiperazine)-5-nitrophenol (12): the mixed solution of 2-fluoro-5-nitrophenol (0.79 g, 5 mmol) and N-methylpiperazine (about 6 ml, 52 mmol) was heated to 90° C. and reacted for more than 5 h. The reaction solution was naturally cooled to room temperature, to which was added water to dilute, and then the precipitation deposited. The resultant solid was filtered by suction, washed with water, and dried to obtain 1.13 g of the product (compound 12), with a yield of 95%. HRMS (ESI⁺) m/z: Calcd for C₁₁H₅N₃O₃: 238.1113 [M+H]⁺; Found 238.1192 [M+H]⁺.

(8) Synthesis of 5-amino-2-(4-methylpiperazine)-phenol (16): compound 12 (0.95 g, 4 mmol) was dissolved in 50 ml isopropanol, mixed, and heated under reflux. Hydrazine hydrate (2 ml, dissolved in 10 ml isopropanol) was added dropwise to the reflux solution, and the mixture was allowed to react for 1 h. The reaction solution was filtered after cooling, and the filtrate was concentrated under reduced pressure to provide 0.81 g of the product (compound 16), with a yield of 98%. HRMS-ESI (m z) calcd for C₁₁H₁₇N₃O: 208.1372 [M+H]⁺; Found 208.1446 [M+H]⁺.

(9) Synthesis of 4-(4-methylpiperazine)-3-nitroaniline (18): the mixed solution of 4-fluoro-3-nitroaniline (7.8 g, 0.05 mol), N-methylpiperazine (28 ml, 0.25 mol), and 30 ml of acetonitrile was allowed to react for more than 3 h at the temperature of 90° C. After completion of the reaction, the solution was naturally cooled to room temperature, and rotatory evaporated to dry. The residue was separated by silica gel column chromatography (eluent: CH₂C₁₂), to provide 11 g of the product as brown solid (compound 18), with a yield of 93%. HRMS-ESI (m/z) calcd for C₁₁H₁₆N₄O₂: 237.1273 [M+H]⁺; Found 237.1351 [M+H]⁺. ¹H NMR (400 MHz, Chloroform-d) δ 7.16-7.11 (m, 1H), 6.82 (d, J=8.4 Hz, 1H), 6.61-6.55 (m, 1H), 3.42 (d, J=32.3 Hz, 2H), 2.86 (d, J=5.0 Hz, 4H), 2.65-2.50 (m, 4H), 2.32 (s, 3H).

### Example 2 Synthesis of Key Intermediate 2

The synthetic route of key intermediate 2 was as follows:

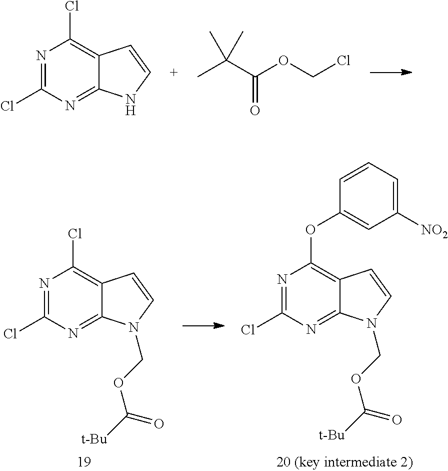

The specific preparative method was as follows:

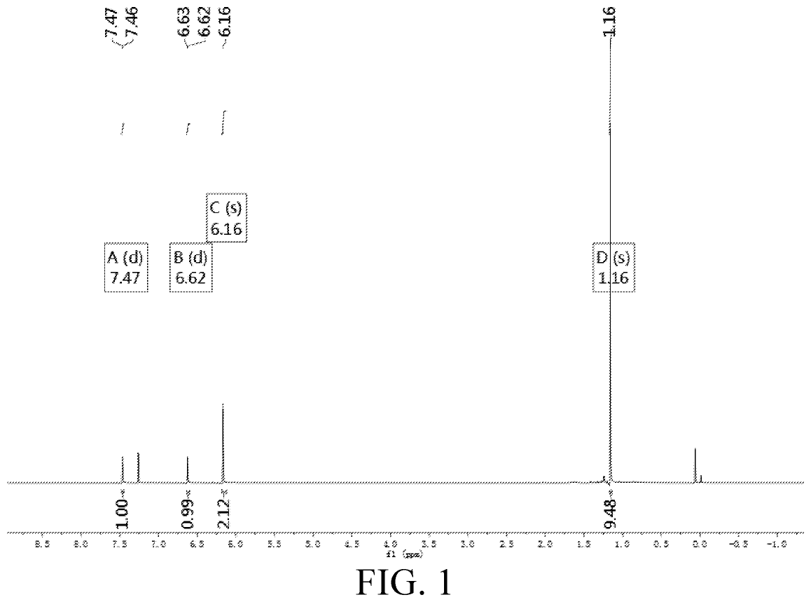

Synthesis of compound 19: 2,4-dichloro-7H-pyrrolo[2,3-d]pyrimidine (4.68 g, 0.025 mol) was added in a round-bottom flask, and dissolved with THF, to which were further added chloromethyl pivalate (7.5 g, 0.05 mol) and K₂CO₃ (0.1 mol, 13.8 g). The mixture was stirred at room temperature for 5 min, and then heated to 80° C. and refluxed. The reaction was further carried out for about 12 h. TLC detection indicated that the reaction was completed (developing solvent: CH₂C₁₂). After it was cooled, the reaction solution was rotatory evaporated to dry, to which were added 150 ml water and CH₂C₁₂ (300 ml×3) for extraction, The organic phases were combined, to which was added a suitable amount of anhydrous sodium sulfate to dry. The solution was filtered and concentrated. The crude product was separated by silica gel column chromatography (eluent: CH₂C₁₂) to obtain 5.43 g of the product as a white solid (compound 19), with a yield of 72%. ¹H NMR spectrum of compound 19 is shown in FIG. 1. HRMS-ESI (m z) calcd for C₁₂H₁₃C₁₂N₃O₂: 302.0385 [M+H]⁺; 324.0385 [M+Na]⁺; Found 302.0463 [M+H]⁺; 324.0278 [M+Na]⁺. ¹H NMR (600 MHz, Chloroform-d): δ 7.47 (d, J=3.8 Hz, 1H), 6.62 (d, J=3.7 Hz, 1H), 6.16 (s, 2H), 1.16 (s, 9H).

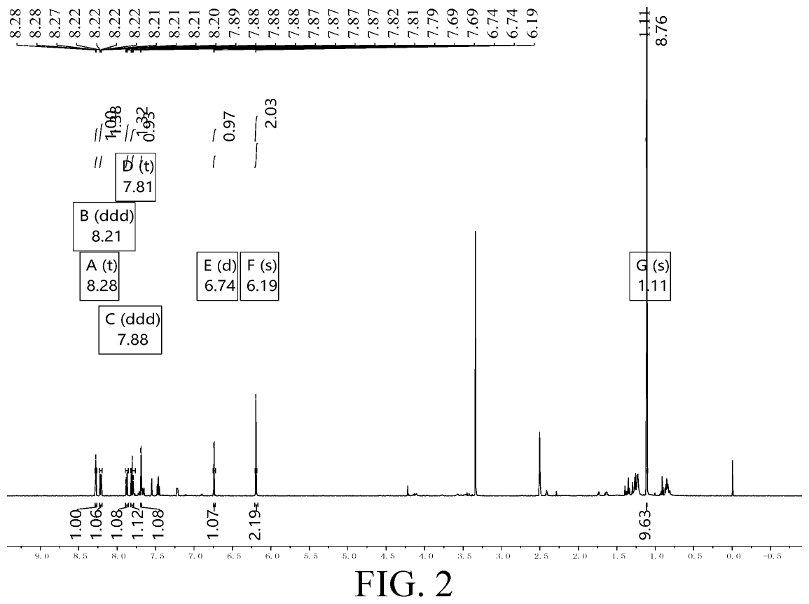

Synthesis of compound 20: 15 g (0.05 mol) of compound 19 was added to a round-bottom flask, and dissolved with acetonitrile, to which were then added 10 g (0.072 mol) of 3-nitrophenol and 13.8 g (1 mol) of K₂CO₃. The mixture was stirred at room temperature for 5 min, and then heated to 80° C. The reaction was further carried out for about 10 h. TLC detection indicated that the reaction was completed (developer: CH₂C₁₂). After it was cooled, the reaction solution was rotatory evaporated to dry, and extracted with the aqueous solution of NaOH (pH=10) and CH₂C₁₂ (300 ml×3) to remove excess 3-nitrophenol. The organic phases were combined, and an appropriate amount of anhydrous sodium sulfate was added for drying. The solution was filtered and concentrated. The crude product was separated by silica gel column chromatography (eluent: CH₂C₁₂) to obtain 14.91 g of the product as a white solid (compound 20), with a yield of 74%. ¹H NMR spectrum of compound 20 is shown in FIG. 2. HRMS (ESI⁺) m/z: calcd for C₁₈H₁₇ClN₄O₅: 427.0887 [M+Na]⁺; Found 427.0779 [M+Na]⁺. ¹H NMR (600 MHz, DMSO-d₆): δ 8.28 (t, J=2.1 Hz, 1H), 8.21 (ddd, J=8.2, 2.2, 1.0 Hz, 1H), 7.88 (ddd, J=8.2, 2.3, 1.0 Hz, 1H), 7.81 (t, J=8.3 Hz, 1H), 6.74 (d, J=3.7 Hz, 1H), 6.19 (s, 2H), 1.11 (s, 9H).

### Example 3 Synthesis of Compound 1 According to the Present Invention

The synthetic route of compound 1 was as follows:

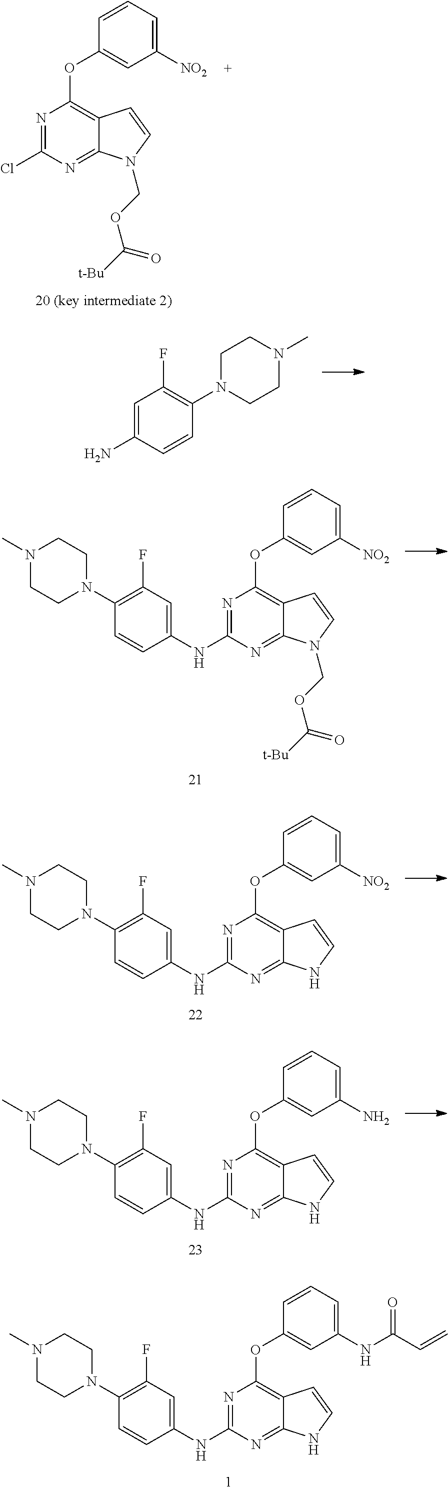 [SMILES: CC(C)(C)C(=O)OCn1ccc2c(Oc3cccc([N+](=O)[O-])c3)nc(Cl)nc21 ; CN1CCN(c2ccc(N)cc2F)CC1 ; CN1CCN(c2ccc(Nc3nc(Oc4cccc([N+](=O)[O-])c4)c4ccn(COC(=O)C(C)(C)C)c4n3)cc2F)CC1 ; CN1CCN(c2ccc(Nc3nc(Oc4cccc([N+](=O)[O-])c4)c4cc[nH]c4n3)cc2F)CC1 ; CN1CCN(c2ccc(Nc3nc(Oc4cccc(N)c4)c4cc[nH]c4n3)cc2F)CC1 ; C=CC(=O)Nc1cccc(Oc2nc(Nc3ccc(N4CCN(C)CC4)c(F)c3)nc3[nH]ccc23)c1]

The specific preparative method was as follows:

Synthesis of compound 21: To a round-bottom flask, were added t-BuOH (50 ml), key intermediate 2 (compound 20) (1.21 g, 3 mmol), and 3-fluoro-4-(4-methylpiperazine)-aniline (522 mg, 2.5 mmol). The above reaction solution was stirred 5-10 min at room temperature, to which were successively added K₂CO₃ (690 mg, 5 mmol), Pd₂(dba)₃ (230 mg, 0.25 mmol, a catalyst, catalyzing the formation of C—N bond), and XPhos (namely 2-(dicyclohexylphosphino)-2,4,6-triisopropylbiphenyl, 120 mg, 0.25 mmol). The reaction solution was stirred and reacted for about 6 h under refluxing at 110° C. TLC detection indicated that the reaction was completed (developing solvent: CH₂Cl₂/CH₃OH=10/1, v/v). After it was cooled to room temperature, the reaction solution was rotatory evaporated to dry, and then subjected to extraction, drying, concentration, and separation by silica gel column chromatography (eluent: CH₂Cl₂/CH₃OH=100:5, v/v), to provide 1.13 g of the product as a red-brown solid (21), with a yield of 78%. HRMS (ESI⁺) m/z: calcd for C₂₉H₃₂FN₇O₅: 578.2449 [M+H]⁺; Found 578.2526 [M+H]⁺.

Synthesis of compound 22: Compound 21 (0.58 g, 1 mmol) was added to a 50 ml round bottom flask, and dissolved in 20 ml methanol. The solution was stirred at room temperature for 5 minutes. The solution of NaOH (0.4 g NaOH was dissolved in 10 ml purified water, and then 4 ml was taken out and used) was added dropwise to the above reaction solution, and the reaction was further stirred and reacted for 5 h. TLC detection indicated that the reaction was completed (developer: CH₂Cl₂/CH₃OH=10/1, v/v). The pH of the solution was adjusted to be neutral. The reaction solution was rotatory evaporated to dry, and then subjected to extraction, drying, and concentration. The crude product was separated by silica gel column chromatography (eluent: CH₂Cl₂/CH₃OH=100:3, v/v), and the separation was performed in two batches, to provide a total of 0.80 g product as a red-brown solid (22), with a yield of 86%. HRMS (ESI⁺) m/z: calcd for C₂₃H₂₂FN₇O₃: 464.1768 [M+H]⁺; Found 464.1846 [M+H]⁺.

Synthesis of compound 23: compound 22 (0.463 g, 1 mmol) and Pd/C (0.08 g) were dissolved in 20 ml isopropanol, mixed, and heated under reflux. Hydrazine hydrate (0.5 ml, dissolved in 10 ml isopropanol) was added dropwise to the reflux solution, and the mixture was allowed to react for 1 h. The reaction was completed by TLC detection (developing solvent: CH₂Cl₂/CH₃OH=10/1, v/v). The reaction mixture was filtered after cooling, and the filtrate was concentrated under reduced pressure to provide 0.42 g of the product (23), with a yield of 97.0%. HRMS (ESI⁺) m/z: calcd for C₂₃H₂₄FN₇O: 434.2026 [M+H]⁺; Found 434.2096 [M+H]⁺.

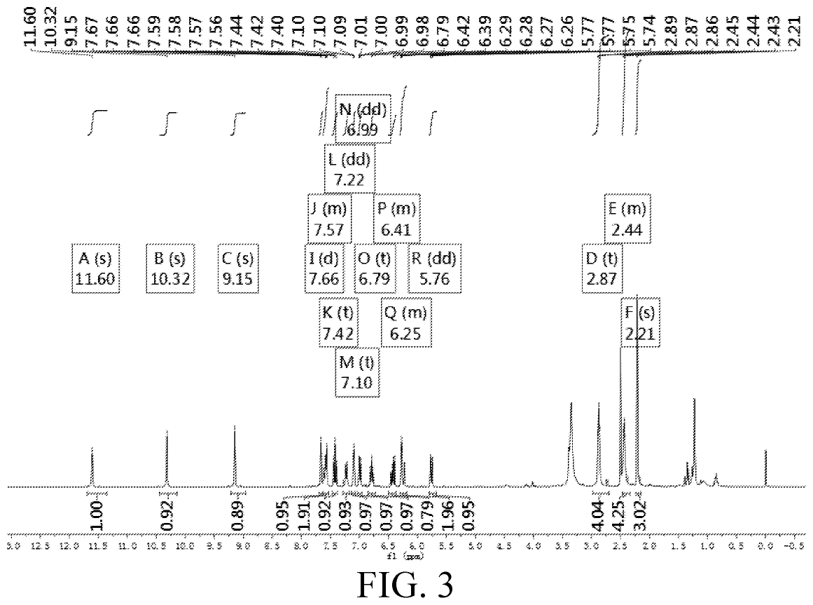

Synthesis of compound 1: compound 23 (0.42 g, 0.943 mmol) was dissolved in 30 ml of THF, to which was added DIEA (312 μl, 1.89 mmol) dropwise, and the reaction device was placed in a low-temperature reactor. When the internal temperature of the reactor reached −3° C., the solution of acryloyl chloride in THE (153 μl, 1.89 mmol) was further added dropwise. The mixture was allowed to react overnight, and the reaction was completed by TLC detection (developer: CH₂Cl₂/CH₃OH=10/1, v/v). The pH of the reaction solution was adjusted to be neutral and in alkaline side with saturated Na₂CO₃ solution. The reaction solution was rotatory evaporated to dry, and then subjected to extraction, drying, concentration, and separation by silica gel column chromatography (eluent: CH₂Cl₂/CH₃OH=100:5, v/v), to provide 0.38 g of solid product (compound 1), with a yield of 83%. ¹H NMR spectrum of compound 1 is shown in FIG. 3. HRMS (ESI⁺) m/z: calcd for C₂₆H₂₆FN₇O₂: 488.2132 [M+H]⁺; Found 488.2206 [M+H]⁺. ¹H NMR (400 MHz, DMSO-d₆) δ 11.62 (s, 1H), 10.32 (s, 1H), 9.16 (s, 1H), 7.78 (d, J=2.5 Hz, 1H), 7.71-7.63 (m, 1H), 7.62-7.55 (m, 1H), 7.51-7.33 (m, 2H), 7.10 (t, J=2.9 Hz, 1H), 6.99 (dd, J=8.1, 2.3 Hz, 1H), 6.90 (d, J=8.8 Hz, 1H), 6.43 (dd, J=16.9, 10.1 Hz, 1H), 6.33-6.19 (m, 2H), 5.76 (dd, J=10.0, 2.0 Hz, 1H), 2.84 (d, J=5.1 Hz, 4H), 2.44 (s, 4H), 2.21 (s, 3H).

### Example 4 Synthesis of Compound 2 According to the Present Invention

The synthetic route of compound 2 was as follows:

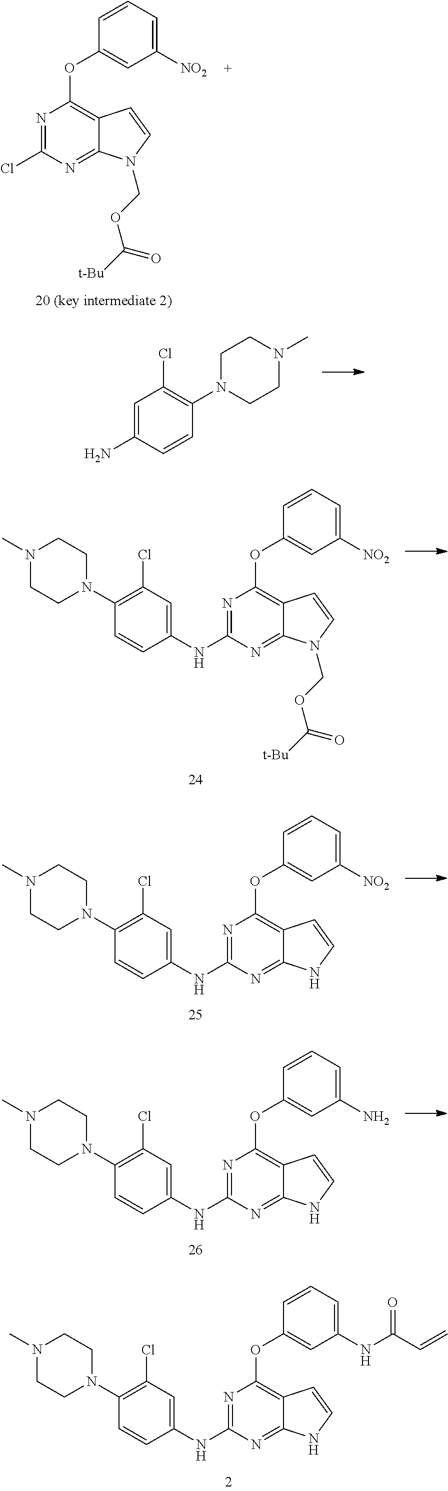 [SMILES: CC(C)(C)C(=O)OCn1ccc2c(Oc3cccc([N+](=O)[O-])c3)nc(Cl)nc21 ; CN1CCN(c2ccc(N)cc2Cl)CC1 ; CN1CCN(c2ccc(Nc3nc(Oc4cccc([N+](=O)[O-])c4)c4ccn(COC(=O)C(C)(C)C)c4n3)cc2Cl)CC1 ; CN1CCN(c2ccc(Nc3nc(Oc4cccc([N+](=O)[O-])c4)c4cc[nH]c4n3)cc2Cl)CC1 ; CN1CCN(c2ccc(Nc3nc(Oc4cccc(N)c4)c4cc[nH]c4n3)cc2Cl)CC1 ; C=CC(=O)Nc1cccc(Oc2nc(Nc3ccc(N4CCN(C)CC4)c(Cl)c3)nc3[nH]ccc23)c1]

The specific preparative method was as follows:

Synthesis of compound 24: To a round-bottom flask, were added t-BuOH (50 ml), key intermediate 2 (compound 20) (1.28 g, 3.17 mmol), and 3-chloro-4-(4-methylpiperazine)-aniline (560 mg, 2.5 mmol). The above reaction solution was stirred 5-10 min at room temperature, to which were successively added K₂CO₃ (690 mg, 5 mmol), Pd₂(dba)₃ (230 mg, 0.25 mmol), and XPhos (120 mg, 0.25 mmol). The reaction solution was stirred and reacted for about 6 h under refluxing at 110° C. TLC detection indicated that the reaction was completed (developing solvent: CH₂Cl₂/CH₃OH=10/1, v/v). After it was cooled to room temperature, the reaction solution was rotatory evaporated to dry, and then subjected to extraction, drying, concentration, and separation by silica gel column chromatography (eluent: CH₂Cl₂/CH₃OH=100:5, v/v), to provide 0.79 g of the product as a red-brown solid (24), with a yield of 54%. HRMS (ESI⁺) m/z: calcd for C₂₉H₃₂ClN₇O₅: 594.2153 [M+H]⁺; Found 594.2228 [M+H]⁺. ¹H NMR (400 MHz, DMSO-d₆) δ 9.50 (s, 1H), 8.35-8.12 (m, 2H), 7.91-7.71 (m, 2H), 7.69 (s, 1H), 7.32 (d, J=3.7 Hz, 1H), 6.88 (d, J=8.8 Hz, 1H), 6.56 (d, J=3.7 Hz, 1H), 6.11 (s, 2H), 2.83 (t, J=4.8 Hz, 4H), 2.43 (s, 4H), 2.21 (s, 3H), 1.11 (s, 9H).

Synthesis of compound 25: Compound 24 (0.59 g, 1 mmol) was added to a 50 ml round bottom flask, and dissolved in 20 ml methanol. The solution was stirred at room temperature for 5 minutes. The solution of NaOH (0.4 g NaOH was dissolved in 10 ml purified water, and then 4 ml was taken out and used) was added dropwise to the above reaction solution, and the reaction was further stirred and reacted for 5 h. TLC detection indicated that the reaction was completed (developer: CH₂Cl₂/CH₃OH=10/1, v/v). The pH of the solution was adjusted to be neutral. The reaction solution was rotatory evaporated to dry, and then subjected to extraction, drying, and concentration. The crude product was separated by silica gel column chromatography (eluent: CH₂Cl₂/CH₃OH=100:3, v/v), to provide 0.91 g of the product as a red-brown solid (25, two batches), with a yield of 95%. HRMS (ESI⁺) m/z: calcd for C₂₃H₂₂ClN₇O₃: 480.1473 [M+H]⁺; Found 480.1551 [M+H]⁺. ¹H NMR (400 MHz, DMSO-d₆) δ 11.69 (s, 1H), 9.21 (s, 1H), 8.20 (dd, J=7.0, 1.9 Hz, 2H), 7.93-7.59 (m, 3H), 7.32 (dd, J=8.9, 2.5 Hz, 1H), 7.16 (dd, J=3.6, 1.7 Hz, 1H), 6.90 (d, J=8.8 Hz, 1H), 6.43 (d, J=3.4 Hz, 1H), 2.83 (t, J=4.8 Hz, 4H), 2.43 (s, 4H), 2.21 (s, 3H).

Synthesis of compound 26: compound 25 (0.479 g, 1 mmol) and Pd/C (0.08 g) were dissolved in 20 ml isopropanol, mixed, and heated under reflux. Hydrazine hydrate (0.5 ml of hydrazine hydrate was dissolved in 10 ml isopropanol) was added dropwise to the reflux solution, and the mixture was allowed to react for 1 h. The reaction was completed by TLC detection (developing solvent: CH₂Cl₂/CH₃OH=10/1, v/v). The reaction mixture was filtered after cooling, and the filtrate was concentrated under reduced pressure to provide 0.43 g of the product (26), with a yield of 96.0%. HRMS (ESI⁺) m/z: calcd for C₂₃H₂₄ClN₇O: 450.1731 [M+H]⁺; Found 450.1806 [M+H]⁺. ¹H NMR (400 MHz, DMSO-d₆) δ 11.54 (d, J=35.6 Hz, 1H), 9.18 (d, J=13.6 Hz, 1H), 7.86 (dd, J=16.5, 2.5 Hz, 1H), 7.73-7.47 (m, 1H), 7.30-6.83 (m, 3H), 6.69-6.26 (m, 3H), 6.14 (ddd, J=27.8, 3.5, 1.9 Hz, 1H), 5.28 (s, 2H), 2.88 (t, J=4.8 Hz, 4H), 2.46 (s, 4H), 2.22 (s, 3H).

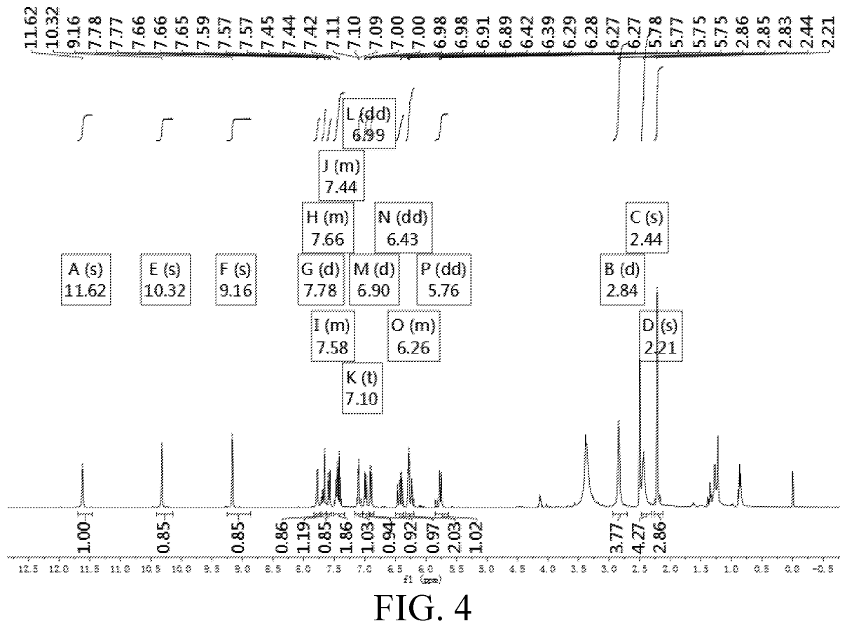

Synthesis of compound 2: compound 26 (0.45 g, 1 mmol) was dissolved in 30 ml of THF, to which was added DIEA (312 μl, 1.89 mmol) dropwise, and the reaction device was placed in a low-temperature reactor. When the internal temperature of the reactor reached −3° C., the solution of acryloyl chloride in THE (153 μl, 1.89 mmol) was further added dropwise. During the dropping process, the temperature was kept between −5° C. and 0° C. The mixture was allowed to react overnight, and the reaction was completed by TLC detection (developer: CH₂Cl₂/CH₃OH=10/1, v/v). The pH of the reaction solution was adjusted to be neutral and in alkaline side with saturated Na₂CO₃ solution. The reaction solution was rotatory evaporated to dry, to which was added 100 ml water and CH₂C₁₂ (150 ml×3) for extraction. The organic phases were combined, and a suitable amount of anhydrous Na₂SO₄ was added to dry. The reaction solution was filtered, concentrated, and separated by silica gel column chromatography (eluent: CH₂Cl₂/CH₃OH=100:5, v/v), to provide 0.41 g of solid product (compound 2), with a yield of 82%. ¹H NMR spectrum of compound 2 is shown in FIG. 4. HRMS (ESI⁺) m/z: calcd for C₂₆H₂₆ClN₇O₂: 504.1837[M+H]⁺; Found 504.1913 [M+H]⁺. ¹H NMR (400 MHz, DMSO-d₆) δ 11.62 (s, 1H), 10.32 (s, 1H), 9.16 (s, 1H), 7.78 (d, J=2.5 Hz, 1H), 7.71-7.63 (m, 1H), 7.62-7.55 (m, 1H), 7.51-7.33 (m, 2H), 7.10 (t, J=2.9 Hz, 1H), 6.99 (dd, J=8.1, 2.3 Hz, 1H), 6.90 (d, J=8.8 Hz, 1H), 6.43 (dd, J=16.9, 10.1 Hz, 1H), 6.33-6.19 (m, 2H), 5.76 (dd, J=10.0, 2.0 Hz, 1H), 2.84 (d, J=5.1 Hz, 4H), 2.44 (s, 4H), 2.21 (s, 3H).

### Example 5 Synthesis of Compound 3 According to the Present Invention

The synthetic route of compound 3 was as follows:

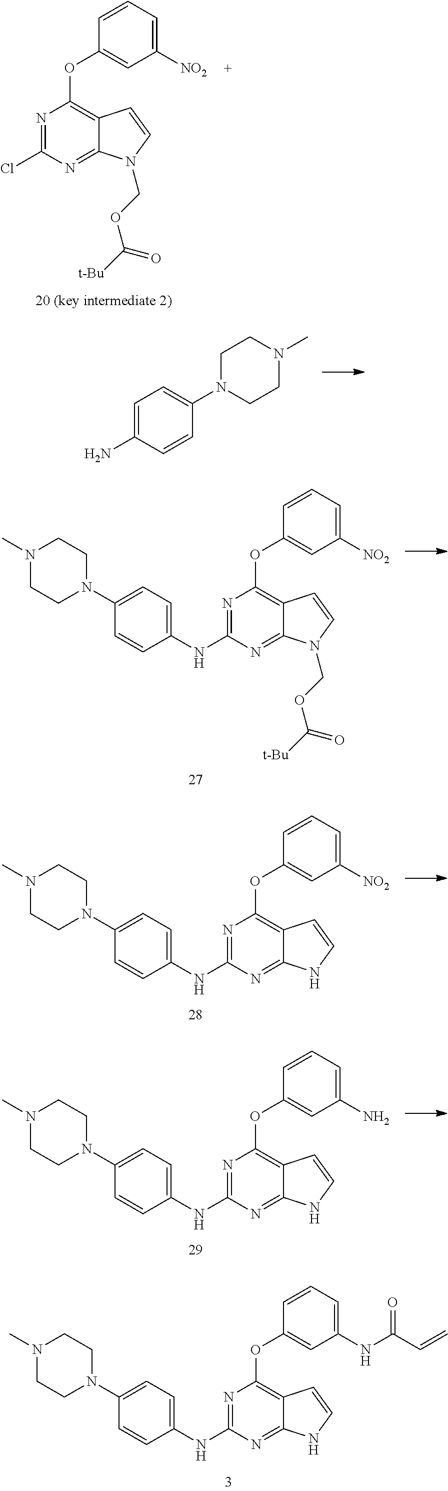 [SMILES: CC(C)(C)C(=O)OCn1ccc2c(Oc3cccc([N+](=O)[O-])c3)nc(Cl)nc21 ; CN1CCN(c2ccc(N)cc2)CC1 ; CN1CCN(c2ccc(Nc3nc(Oc4cccc([N+](=O)[O-])c4)c4ccn(COC(=O)C(C)(C)C)c4n3)cc2)CC1 ; CN1CCN(c2ccc(Nc3nc(Oc4cccc([N+](=O)[O-])c4)c4cc[nH]c4n3)cc2)CC1 ; CN1CCN(c2ccc(Nc3nc(Oc4cccc(N)c4)c4cc[nH]c4n3)cc2)CC1 ; C=CC(=O)Nc1cccc(Oc2nc(Nc3ccc(N4CCN(C)CC4)cc3)nc3[nH]ccc23)c1]

The specific preparative method was as follows:

Synthesis of compound 27: To a round-bottom flask, were added t-BuOH (50 ml), key intermediate 2 (compound 20) (0.404 g, 1 mmol), and intermediate (4-methylpiperazine)aniline (191 mg, 1 mmol). The above reaction solution was stirred 5-10 min at room temperature, to which were successively added K₂CO₃ (276 mg, 2 mmol), Pd₂(dba)₃ (18 mg, 0.02 mmol), and XPhos (19 mg, 0.04 mmol). The reaction solution was stirred and reacted for about 6 h under refluxing at 110° C. TLC detection indicated that the reaction was completed (developing solvent: CH₂Cl₂/CH₃OH=10/1, v/v). After it was cooled to room temperature, the reaction solution was rotatory evaporated to dry, and then subjected to extraction, drying, concentration, and separation by silica gel column chromatography (eluent: CH₂Cl₂/CH₃OH=100:5, v/v), to provide 0.397 g of the product as a red-brown solid (27), with a yield of 71%. HRMS (ESI⁺) m/z: calcd for C₂₉H₃₃N₇O₅: 560.2543 [M+H]⁺; Found 560.2620 [M+H]⁺. ¹H NMR (400 MHz, Chloroform-d) δ 8.12 (dt, J=8.7, 2.4 Hz, 2H), 7.62-7.52 (m, 2H), 7.35 (s, 1H), 7.03 (d, J=3.7 Hz, 1H), 6.91 (s, 1H), 6.82-6.74 (m, 2H), 6.46 (d, J=3.7 Hz, 1H), 6.09 (s, 2H), 3.44 (s, 2H), 3.13 (t, J=4.9 Hz, 4H), 2.60 (t, J=5.0 Hz, 4H), 2.36 (s, 3H), 1.18 (s, 9H).

Synthesis of compound 28: Compound 27 (0.28 g, 0.5 mmol) was added to a 50 ml round bottom flask, and dissolved in 10 ml methanol. The solution was stirred at room temperature for 5 minutes. The solution of NaOH (0.4 g NaOH was dissolved in 10 ml purified water, and then 2 ml was taken out and used) was added dropwise to the above reaction solution, and the reaction was further stirred and reacted for 5 h. TLC detection indicated that the reaction was completed (developer: CH₂Cl₂/CH₃OH=10/1, v/v). The pH of the solution was adjusted to be neutral. The reaction solution was rotatory evaporated to dry, and then subjected to extraction, drying, and concentration. The crude product was separated by silica gel column chromatography (eluent: CH₂Cl₂/CH₃OH=100:3, v/v), to provide 0.14 g of the product as a red-brown solid (28), with a yield of 63%. HRMS (ESI⁺) m/z: calcd for C₂₃H₂₃N₇O₃: 446.1862 [M+H]⁺; Found 446.1940 [M+H]⁺. ¹H NMR (400 MHz, DMSO-d₆) δ 11.71-11.08 (m, 1H), 8.83 (s, 1H), 7.69-7.33 (m, 2H), 7.07 (t, J=8.0 Hz, 1H), 6.98 (dd, J=3.5, 2.2 Hz, 1H), 6.81-6.67 (m, 2H), 6.47 (dd, J=8.0, 2.1 Hz, 1H), 6.40 (t, J=2.2 Hz, 1H), 6.35 (dd, J=7.9, 2.2 Hz, 1H), 6.06 (dd, J=3.5, 1.9 Hz, 1H), 5.27 (s, 2H), 3.01 (t, J=5.0 Hz, 4H), 2.44 (d, J=5.2 Hz, 4H), 2.22 (s, 3H).

Synthesis of compound 29: compound 28 (0.15 g, 0.33 mmol) and Pd/C (0.02 g) were dissolved in 10 ml isopropanol, mixed, and heated under reflux. Hydrazine hydrate (0.2 ml of hydrazine hydrate was dissolved in 3 ml isopropanol) was added dropwise to the reflux solution, and the mixture was allowed to react for 1 h. The reaction was completed by TLC detection (developing solvent: CH₂Cl₂/CH₃OH=10/1, v/v). The reaction mixture was filtered after cooling, and the filtrate was concentrated under reduced pressure to provide 94 mg of the product (29), with a yield of 69%. HRMS (ESI⁺) m/z: calcd for C₂₃H₂₅N₇O: 416.2121 [M+H]⁺; Found 416.2195 [M+H]⁺.

Synthesis of compound 3: compound 29 (0.05 g, 0.12 mmol) was dissolved in 30 ml of THF, to which was added DIEA (40 μl, 0.24 mmol) dropwise, and the reaction device was placed in a low-temperature reactor. When the internal temperature of the reactor reached −3° C., the solution of acryloyl chloride in THE (20 μl, 0.24 mmol) was added dropwise. The mixture was allowed to react overnight, and the reaction was completed by TLC detection (developer: CH₂Cl₂/CH₃OH=10/1, v/v).

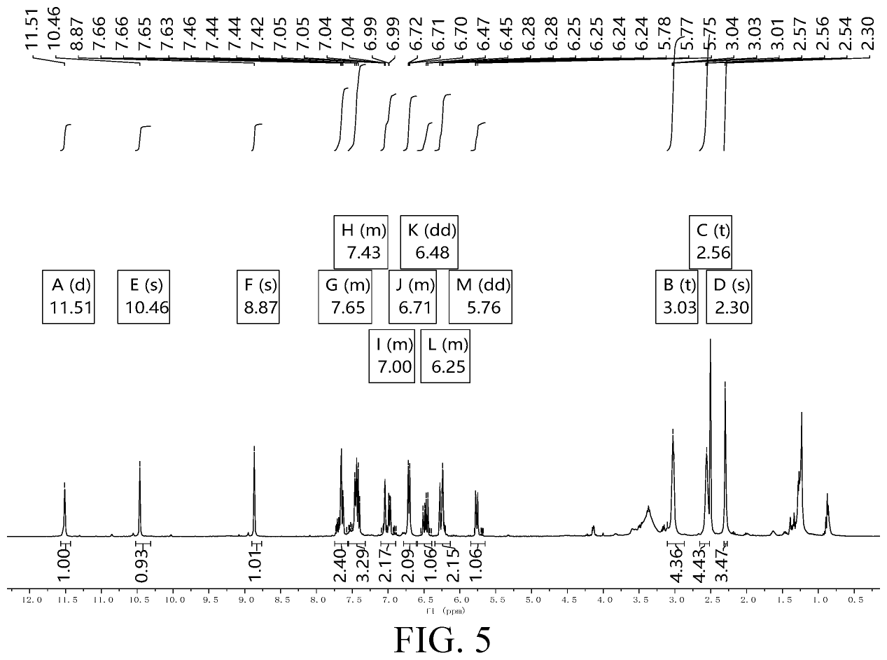

The pH of the reaction solution was adjusted to be neutral and in alkaline side with saturated Na₂CO₃ solution. The reaction solution was rotatory evaporated to dry, to which was added 100 ml water and CH₂C₁₂ (150 ml×3) for extraction. The organic phases were combined, and a suitable amount of anhydrous Na₂SO₄ was added to dry. The reaction solution was filtered, concentrated, and separated by silica gel column chromatography (eluent: CH₂Cl₂/CH₃OH=100:5, v/v), to provide 32.2 mg of red-brown product (compound 3), with a yield of 57%. ¹H NMR spectrum of compound 3 is shown in FIG. 5. HRMS (ESI⁺) m/z: calcd for C₂₆H₂₇N₇O₂: 470.2226 [M+H]⁺; Found 470.2295 [M+H]⁺. ¹H NMR (400 MHz, DMSO-d₆) δ 11.51 (d, J=2.3 Hz, 1H), 10.46 (s, 1H), 8.87 (s, 1H), 7.75-7.56 (m, 2H), 7.55-7.32 (m, 3H), 7.10-6.89 (m, 2H), 6.78-6.61 (m, 2H), 6.48 (dd, J=17.0, 10.1 Hz, 1H), 6.35-6.13 (m, 2H), 5.76 (dd, J=10.1, 2.1 Hz, 1H), 3.03 (t, J=5.0 Hz, 4H), 2.56 (t, J=5.1 Hz, 4H), 2.30 (s, 3H).

### Example 6 Synthesis of Compound 4 According to the Present Invention

The synthetic route of compound 4 was as follows:

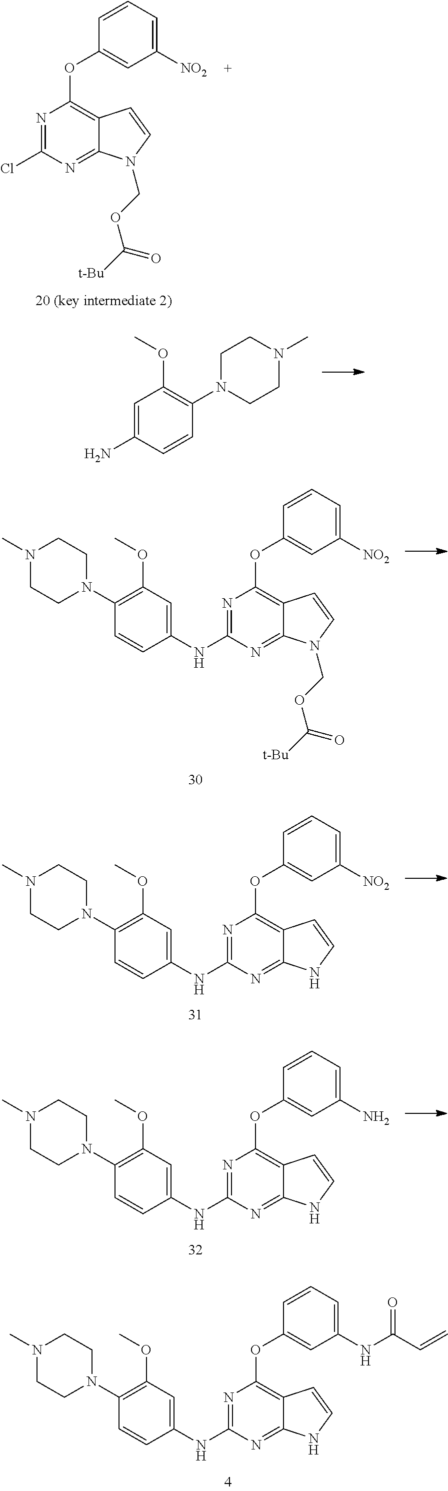 [SMILES: CC(C)(C)C(=O)OCn1ccc2c(Oc3cccc([N+](=O)[O-])c3)nc(Cl)nc21 ; COc1cc(N)ccc1N1CCN(C)CC1 ; COc1cc(Nc2nc(Oc3cccc([N+](=O)[O-])c3)c3ccn(COC(=O)C(C)(C)C)c3n2)ccc1N1CCN(C)CC1 ; COc1cc(Nc2nc(Oc3cccc([N+](=O)[O-])c3)c3cc[nH]c3n2)ccc1N1CCN(C)CC1 ; COc1cc(Nc2nc(Oc3cccc(N)c3)c3cc[nH]c3n2)ccc1N1CCN(C)CC1 ; C=CC(=O)Nc1cccc(Oc2nc(Nc3ccc(N4CCN(C)CC4)c(OC)c3)nc3[nH]ccc23)c1]

The specific preparative method was as follows:

Synthesis of compound 30: To a round-bottom flask, were added t-BuOH (50 ml), key intermediate 2 (compound 20) (0.404 g, 1 mmol), and methoxy intermediate 17 (221 mg, 1 mmol). The above reaction solution was stirred 5-10 min at room temperature, to which were successively added K₂CO₃ (276 mg, 2 mmol), Pd₂(dba)₃ (18 mg, 0.02 mmol), and XPhos (19 mg, 0.04 mmol). The reaction solution was stirred and reacted for about 6 h under refluxing at 110° C. TLC detection indicated that the reaction was completed (developing solvent: CH₂Cl₂/CH₃OH=100/10, v/v). After it was cooled to room temperature, the reaction solution was rotatory evaporated to dry, and then subjected to extraction, drying, concentration, and separation by silica gel column chromatography (eluent: CH₂Cl₂/CH₃OH=100:5, v/v), to provide 0.44 g of the product as a red-brown solid (30), with a yield of 75%. HRMS (ESI⁺) m/z: calcd for C₃₀H₃₅N₇O₆: 590.2649 [M+H]⁺; Found 590.2729 [M+H]⁺. ¹H NMR (400 MHz, Chloroform-d) δ 8.17-8.00 (m, 2H), 7.68-7.53 (m, 2H), 7.52-7.44 (m, 1H), 7.07 (d, J=3.7 Hz, 1H), 6.92-6.84 (m, 2H), 6.80 (d, J=8.6 Hz, 1H), 6.46 (d, J=3.6 Hz, 1H), 6.11 (s, 2H), 3.81 (s, 3H), 3.07 (s, 4H), 2.68 (s, 4H), 2.39 (s, 3H), 1.17 (s, 9H).

Synthesis of compound 31: Compound 30 (0.48 g, 1 mmol) was added to a 50 ml round bottom flask, and dissolved in 10 ml methanol. The solution was stirred at room temperature for 5 minutes. The solution of NaOH (0.4 g NaOH was dissolved in 10 ml purified water, and then 4 ml was taken out and used) was added dropwise to the above reaction solution, and the reaction was further stirred and reacted for 5 h. TLC detection indicated that the reaction was completed (developer: CH₂Cl₂/CH₃OH=10/3, v/v). The pH of the solution was adjusted to be neutral. The reaction solution was rotatory evaporated to dry, and then subjected to extraction, drying, and concentration. The crude product was separated by silica gel column chromatography (eluent: CH₂Cl₂/CH₃OH=100:3, v/v), to provide 0.27 g of the product as a red-brown solid (31), with a yield of 57%. HRMS (ESI⁺) m/z: calcd for C₂₄H₂₅N₇O₄: 476.1968 [M+H]⁺; Found 476.2046 [M+H]⁺. ¹H NMR (600 MHz, DMSO-d₆) δ 11.70-11.55 (m, 1H), 8.92 (s, 1H), 8.23-8.08 (m, 2H), 7.87-7.68 (m, 2H), 7.39-7.25 (m, 1H), 7.18-7.00 (m, 2H), 6.62 (d, J=8.6 Hz, 1H), 6.38 (dd, J=3.5, 1.9 Hz, 1H), 3.64 (s, 3H), 2.91 (s, 4H), 2.61 (s, 4H), 2.33 (s, 3H).

Synthesis of compound 32: compound 31 (0.15 g, 1.07 mmol) and Pd/C (0.08 g) were dissolved in 30 ml isopropanol, mixed, and heated under reflux. Hydrazine hydrate (0.5 ml of hydrazine hydrate was dissolved in 10 ml isopropanol) was added dropwise to the reflux solution, and the mixture was allowed to react for 1 h. The reaction was completed by TLC detection (developing solvent: CH₂Cl₂/CH₃OH=10/1, v/v). The reaction mixture was filtered after cooling, and the filtrate was concentrated under reduced pressure to provide 0.34 g of the product (32), with a yield of 71%. HRMS (ESI⁺) m/z: calcd for C₂₄H₂₇N₇O₂: 446.2226 [M+H]⁺; Found 446.2304 [M+H]⁺. ¹H NMR (400 MHz, DMSO-d₆) δ 11.46 (d, J=2.2 Hz, 1H), 8.88 (s, 1H), 7.78 (t, J=5.3 Hz, 1H), 7.46 (d, J=2.3 Hz, 1H), 7.22 (dd, J=8.6, 2.4 Hz, 1H), 7.08-6.96 (m, 2H), 6.70 (d, J=8.7 Hz, 1H), 6.49-6.26 (m, 3H), 6.09 (dd, J=3.5, 1.9 Hz, 1H), 5.25 (s, 2H), 3.66 (s, 3H), 2.90 (s, 4H), 2.55 (s, 3H), 2.32-2.25 (m, 4H).

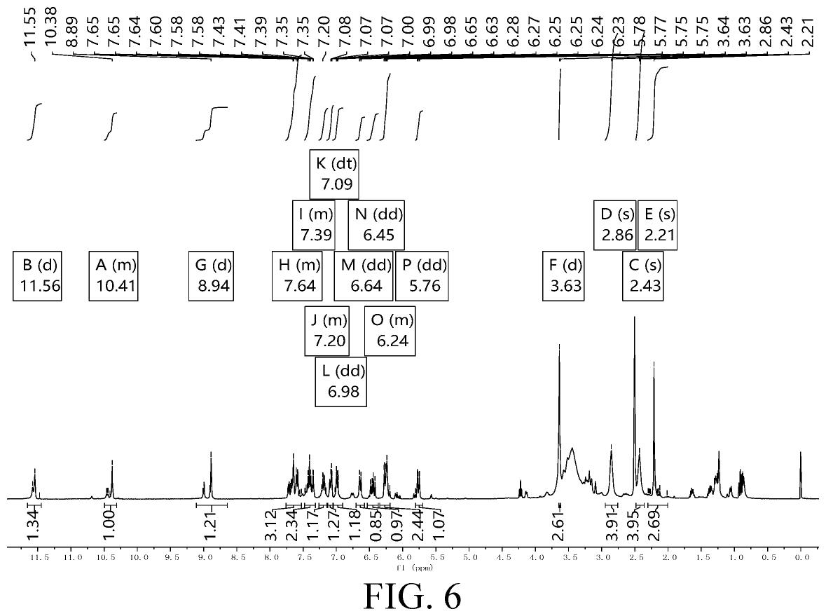

Synthesis of compound 4: compound 32 (0.42 g, 0.943 mmol) was dissolved in 30 ml of THF, to which was added DIEA (312 μl, 1.89 mmol) dropwise, and the reaction device was placed in a low-temperature reactor. When the internal temperature of the reactor reached −3° C., the solution of acryloyl chloride in THE (153 μl, 1.89 mmol) was added dropwise. The mixture was allowed to react overnight, and the reaction was completed by TLC detection (developer: CH₂Cl₂/CH₃OH=10/1, v/v). The pH of the reaction solution was adjusted to be neutral and in alkaline side with saturated Na₂CO₃ solution. The reaction solution was rotatory evaporated to dry, to which was added 50 ml water and CH₂C₂ (50 ml×3) for extraction. The organic phases were combined, and a suitable amount of anhydrous Na₂SO₄ was added to dry. The reaction solution was filtered, concentrated, and separated by silica gel column chromatography (eluent: CH₂Cl₂/CH₃OH=100:5, v/v), to provide 0.254 g of red-brown product (compound 4), with a yield of 54%. ¹H NMR spectrum of compound 4 is shown in FIG. 6. HRMS (ESI⁺) m/z: calcd for C₂₇H₂₉N₇O₃: 500.2332 [M+H]⁺; Found 500.2415 [M+H]⁺. ¹H NMR (400 MHz, DMSO-d₆) δ 11.56 (d, J=13.2 Hz, 1H), 10.50-10.31 (m, 1H), 8.94 (d, J=43.4 Hz, 1H), 7.76-7.53 (m, 3H), 7.48-7.32 (m, 2H), 7.26-7.13 (m, 1H), 7.09 (dt, J=9.6, 2.8 Hz, 1H), 6.98 (dd, J=8.1, 2.4 Hz, 1H), 6.64 (dd, J=8.9, 4.5 Hz, 1H), 6.45 (dd, J=17.0, 10.0 Hz, 1H), 6.35-6.19 (m, 2H), 5.76 (dd, J=10.0, 2.1 Hz, 1H), 3.63 (d, J=4.7 Hz, 3H), 2.86 (s, 4H), 2.43 (s, 4H), 2.21 (s, 3H).

### Example 7. Synthesis of Compounds 5 and 6 According to the Present Invention

The synthetic route for compounds 5 and 6 was as follows:

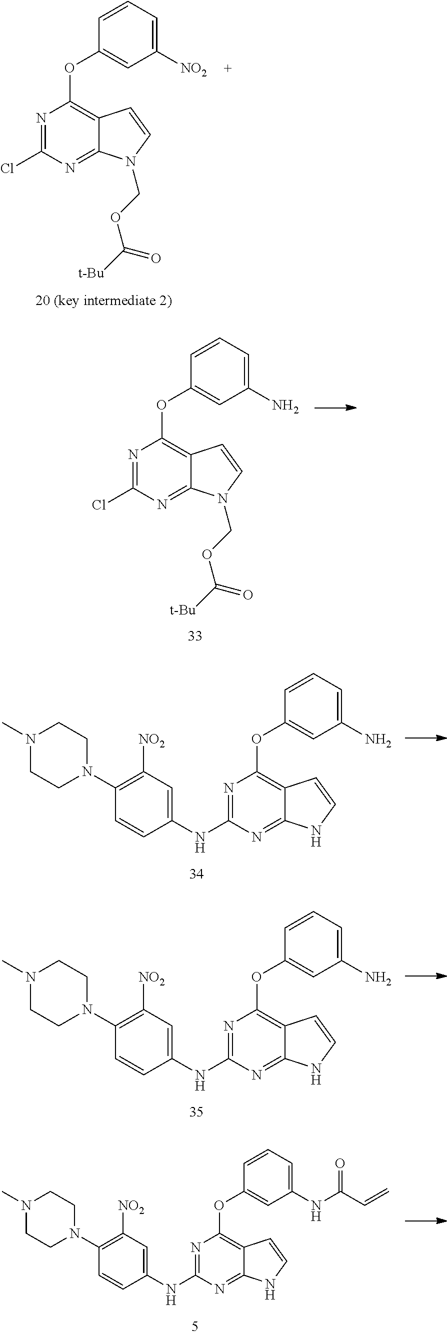 [SMILES: CC(C)(C)C(=O)OCn1ccc2c(Oc3cccc([N+](=O)[O-])c3)nc(Cl)nc21 ; CC(C)(C)C(=O)OCn1ccc2c(Oc3cccc(N)c3)nc(Cl)nc21 ; CN1CCN(c2ccc(Nc3nc(Oc4cccc(N)c4)c4cc[nH]c4n3)cc2[N+](=O)[O-])CC1 ; CN1CCN(c2ccc(Nc3nc(Oc4cccc(N)c4)c4cc[nH]c4n3)cc2[N+](=O)[O-])CC1 ; C=CC(=O)Nc1cccc(Oc2nc(Nc3ccc(N4CCN(C)CC4)c([N+](=O)[O-])c3)nc3[nH]ccc23)c1]

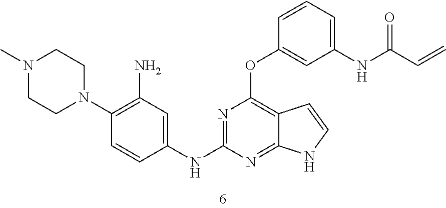 [SMILES: C=CC(=O)Nc1cccc(Oc2nc(Nc3ccc(N4CCN(C)CC4)c(N)c3)nc3[nH]ccc23)c1]

The specific preparative method was as follows:

Synthesis of compound 33: key intermediate 2 (compound 20) (0.68 g, 1.68 mmol) was dissolved in 30 ml methanol, to which were added SnCl₂ (1.57 g, 8.3 mmol) and 4 drops of concentrated hydrochloric acid. The reaction solution was heated under refluxing at 65° C. After reaction for 6 h under refluxing, TLC detection indicated that the reaction was completed (developing solvent: CH₂Cl₂/CH₃OH=100/5, v/v). The pH of the reaction solution was adjusted to be neutral and in alkaline side with saturated Na₂CO₃ solution, and the solution became turbid. The reaction solution was rotatory evaporated to dry, and then extracted with CH₂C₁₂, to provide 0.603 g of the product as a white solid (33), with a yield of 96%. HRMS (ESI⁺) m/z: calcd for C₁₈H₁₉ClN₄O₃: 375.1146 [M+H]⁺, 397.1034 [M+Na]⁺; Found 375.1220 [M+H]⁺, 397.1038 [M+Na]⁺. ¹H NMR (400 MHz, DMSO-d₆) δ 7.57 (d, J=3.7 Hz, 1H), 7.08 (t, J=8.0 Hz, 1H), 6.55-6.48 (m, 2H), 6.44-6.33 (m, 4H), 6.14 (s, 2H), 5.37 (s, 2H), 1.09 (s, 9H).

Synthesis of compound 34: To a round-bottom flask, were added t-BuOH (50 ml), compound 33 (0.56 g, 1.5 mmol), and 4-(4-methylpiperazine)-3-nitroaniline (18) (0.35 g, 1.5 mmol). The above reaction mixture was stirred 5-10 min at a speed of 360 rpm, to which were successively added K₂CO₃ (0.69 g, 3 mmol), Pd₂(dba)₃ (28 mg, 0.03 mmol), and XPhos (29 mg, 0.06 mmol). The reaction solution was stirred and reacted for about 6 h under refluxing at 110° C. TLC detection indicated that the reaction was completed (developing solvent: CH₂Cl₂/CH₃OH=100/10, v/v). After it was cooled to room temperature, the reaction solution was rotatory evaporated to dry, and then subjected to extraction, drying, concentration, and separation by silica gel column chromatography (eluent: CH₂Cl₂/CH₃OH=100:5, v/v), to provide 0.46 g of red-brown solid (34), with a yield of about 53%. HRMS (ESI⁺) m/z: calcd for C₂₉H₃₄N₈O₅: 575.2652 [M+H]⁺; Found 575.2730 [M+H]⁺.

Synthesis of compound 35: Compound 34 (0.46 g, 0.8 mmol) was dissolved in 10 ml methanol, and stirred at room temperature for 5 minutes. The solution of NaOH (0.4 g NaOH was dissolved in 10 ml purified water, and then 4 ml was taken out) was added dropwise to the reactor, and the reaction was further stirred and reacted for 5 h. TLC detection indicated that the reaction was completed (developer: CH₂Cl₂/CH₃OH=10/1, v/v). 0.28 g of red-brown solid (35) was obtained, with a yield of about 76%. HRMS (ESI⁺)m/z: calcd for C₂₃H₂₄N₈O₃: 461.1971 [M+H]⁺; Found 461.2041 [M+H]⁺. ¹H NMR (400 MHz, DMSO-d₆) δ 11.81-11.32 (m, 1H), 9.60-8.87 (m, 1H), 8.31-8.05 (m, 1H), 7.91-7.45 (m, 1H), 7.29-6.90 (m, 3H), 6.77-6.04 (m, 4H), 5.26 (d, J=15.1 Hz, 1H), 2.96-2.65 (m, 3H), 2.37 (dt, J=26.1, 4.8 Hz, 3H), 2.19 (d, J=9.9 Hz, 2H).

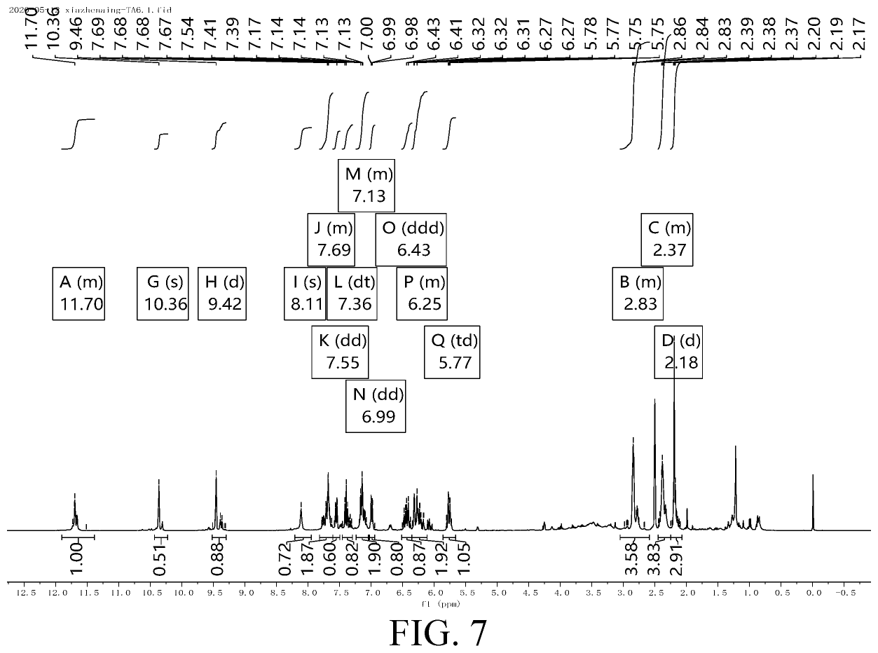

Synthesis of compound 5: compound 35 (250 mg, 0.54 mmol) was dissolved in 30 ml of THF, to which was added DIEA (178 μl, 1.08 mmol) dropwise, and the reaction device was placed in a low-temperature reactor. When the internal temperature of the reactor reached −3° C., the solution of acryloyl chloride in THF (87 μl, 1.08 mmol) was added dropwise. During the dropping process, the temperature was kept between −5° C. and 0° C. The mixture was allowed to react overnight, and the reaction was completed by TLC detection (developer: CH₂Cl₂/CH₃OH=10/1, v/v). The pH of the reaction solution was adjusted to be neutral and in alkaline side with saturated Na₂CO₃ solution. The reaction solution was rotatory evaporated to dry, to which was added 100 ml water and CH₂C₁₂ (150 ml×3) for extraction. The organic phases were combined, and a suitable amount of anhydrous Na₂SO₄ was added to dry. The reaction solution was filtered, concentrated, and separated by silica gel column chromatography (eluent: CH₂Cl₂/CH₃OH=100:5, v/v). The reaction was carried out in two batches, and a total of 0.42 g product (compound 5) was obtained, with a yield of 76%. ¹H NMR spectrum of compound 5 is shown in FIG. 7. HRMS (ESI⁺) m/z: calcd for C₂₆H₂₆N₈O₄ 515.2077 [M+H]⁺; Found 515.2156 [M+H]⁺. ¹H NMR (400 MHz, DMSO-d₆) δ 11.90-11.39 (m, 1H), 10.36 (s, 1H), 9.42 (d, J=26.9 Hz, 1H), 8.11 (s, 1H), 7.82-7.61 (m, 2H), 7.55 (dd, J=8.2, 1.9 Hz, 1H), 7.36 (dt, J=25.9, 8.1 Hz, 1H), 7.23-7.04 (m, 2H), 6.99 (dd, J=8.0, 2.4 Hz, 1H), 6.43 (ddd, J=16.9, 12.9, 10.1 Hz, 1H), 6.35-6.11 (m, 2H), 5.77 (td, J=10.4, 2.1 Hz, 1H), 3.05-2.59 (m, 4H), 2.45-2.25 (m, 4H), 2.18 (d, J=7.7 Hz, 3H).

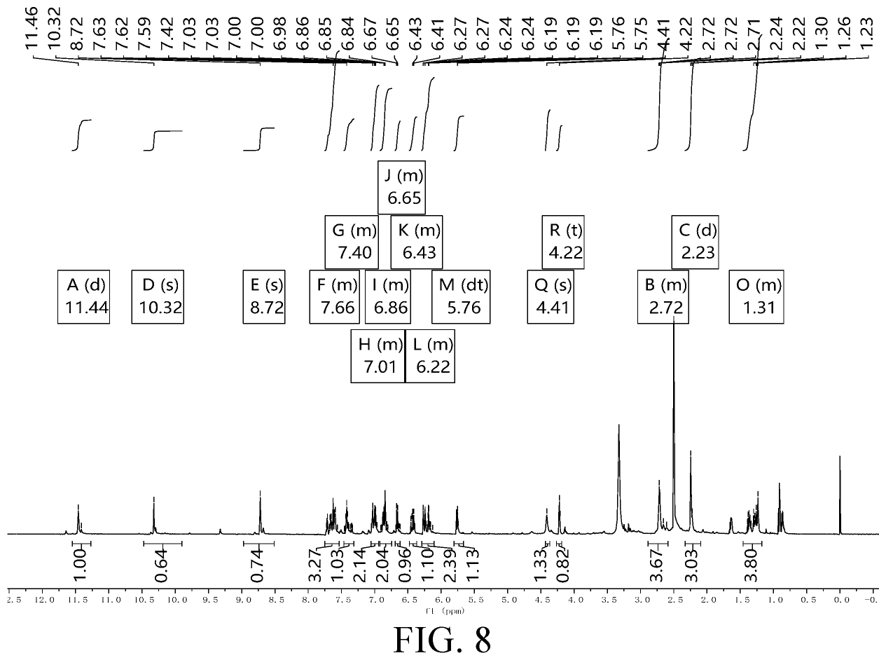

Synthesis of compound 6: compound 5 (300 mg, 0.58 mmol) was dissolved in 30 ml methanol, to which were then added SnCl₂ (600 mg, 3.16 mmol) and 10 drops of concentrated hydrochloric acid. The reaction solution was heated under refluxing at 65° C. After reaction for 6 h under refluxing, TLC detection indicated that the reaction was completed (developing solvent: CH₂Cl₂/CH₃OH=5/1). The pH of the reaction solution was adjusted to be neutral and in alkaline side with saturated Na₂CO₃ solution, and then the solution was extracted with CH₂C₁₂, to provide about 140 mg of the product as a brown solid (compound 6), with a yield of 50%. ¹H NMR spectrum of compound 6 is shown in FIG. 8. HRMS (ESI⁺) m/z: calcd for C₂₆H₂₈N₈O₂ 485.2335 [M+H]⁺; Found 485.2407 [M+H]⁺. ¹H NMR (600 MHz, DMSO-d₆) δ 11.44 (d, J=27.0 Hz, 1H), 10.32 (s, 1H), 8.72 (s, 1H), 7.75-7.53 (m, 3H), 7.46-7.31 (m, 1H), 7.06-6.94 (m, 2H), 6.92-6.74 (m, 2H), 6.69-6.62 (m, 1H), 6.48-6.37 (m, 1H), 6.29-6.11 (m, 2H), 5.76 (dt, J=12.1, 4.5 Hz, 1H), 4.41 (s, 1H), 4.22 (t, J=6.6 Hz, 1H), 2.89-2.59 (m, 4H), 2.23 (d, J=11.9 Hz, 3H), 1.46-1.18 (m, 4H).

### Example 8 Synthesis of Compounds 7 to 9 According to the Present Invention

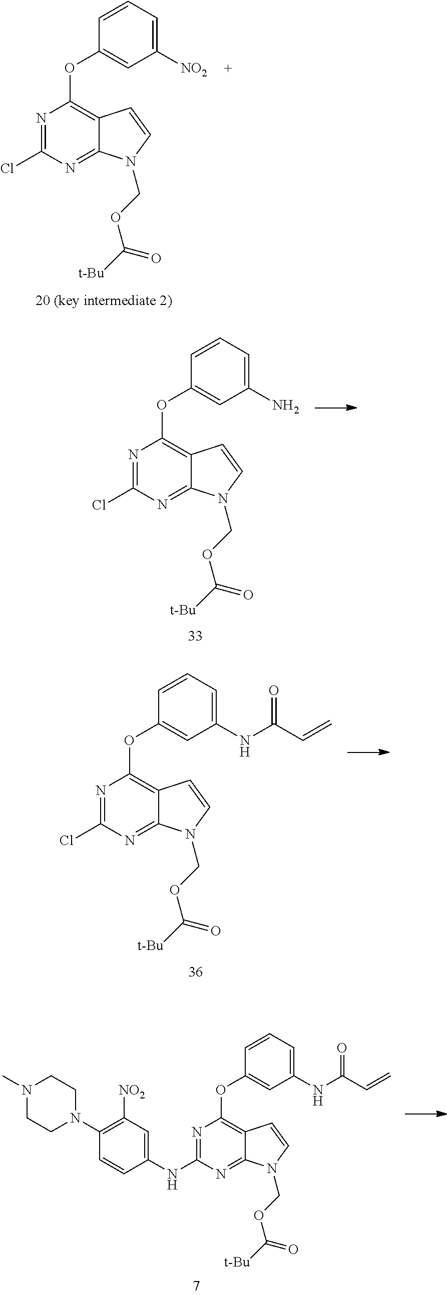

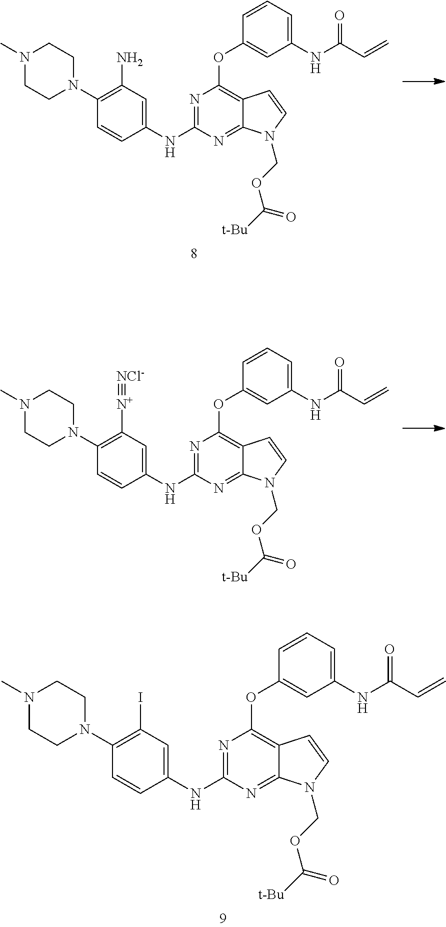

The specific preparative method was as follows:

Synthesis of compound 33: key intermediate 2 (compound 20) (6.06 g, 15 mmol) was dissolved in 150 ml methanol, to which were added SnCl₂ (14 g, 75 mmol) and 30 drops of concentrated hydrochloric acid. The reaction solution was heated under refluxing at 65° C. After reaction for 6 h under refluxing, TLC detection indicated that the reaction was completed (developing solvent: CH₂Cl₂/CH₃OH=100/5, v/v). The pH of the reaction solution was adjusted to be neutral and in alkaline side with saturated Na₂CO₃ solution, and the solution became turbid. The reaction solution was rotatory evaporated to dry, and then extracted with CH₂C₁₂, to provide 5.43 g of the product as a white solid (33), with a yield of 97%. HRMS (ESI⁺) m/z: calcd for C₁₈H₁₉ClN₄O₃: 375.1146 [M+H]⁺, 397.1034 [M+Na]⁺; Found 375.1220 [M+H]⁺, 397.1038 [M+Na]⁺. ¹H NMR (400 MHz, DMSO-d₆) δ 7.57 (d, J=3.7 Hz, 1H), 7.08 (t, J=8.0 Hz, 1H), 6.55-6.48 (m, 2H), 6.44-6.33 (m, 4H), 6.14 (s, 2H), 5.37 (s, 2H), 1.09 (s, 9H).

Synthesis of compound 36: compound 33 (5.61 g, 15 mmol) was dissolved in 50 ml of THF, to which was added DIEA (4.96 ml, 30 mmol) dropwise, and the reaction device was placed in a low-temperature reactor. When the internal temperature of the reactor reached −3° C., the solution of acryloyl chloride in THE (2.43 ml, 30 mmol) was added dropwise. The mixture was allowed to react overnight, and the reaction was completed by TLC detection (developer: CH₂Cl₂/CH₃OH=10/1, v/v). The pH of the reaction solution was adjusted to be neutral and in alkaline side with saturated Na₂CO₃ solution. The reaction solution was rotatory evaporated to dry, to which was added 100 ml water and CH₂C₁₂ (150 ml×3) for extraction. The organic phases were combined, and a suitable amount of anhydrous Na₂SO₄ was added to dry. The reaction solution was filtered, concentrated, and separated by silica gel column chromatography (eluent: CH₂Cl₂/CH₃OH=100:5, v/v), to provide 10.42 g of the product as a white solid (in two batches), with a yield of 81%. HRMS (ESI⁺) m/z: calcd for C₂₁H₂₁ClN₄O₄: 451.1251 [M+Na]⁺; Found 451.1140 [M+Na]⁺. ¹H NMR (400 MHz, Chloroform-d) δ 7.93 (s, 1H), 7.74 (s, 1H), 7.27 (d, J=3.7 Hz, 1H), 7.19 (d, J=8.0 Hz, 1H), 7.10 (d, J=8.7 Hz, 1H), 6.83 (d, J=9.1 Hz, 1H), 6.50 (d, J=3.7 Hz, 1H), 6.29 (d, J=16.8 Hz, 1H), 6.14-6.01 (m, 3H), 5.60 (d, J=11.1 Hz, 1H), 1.13 (s, 9H).

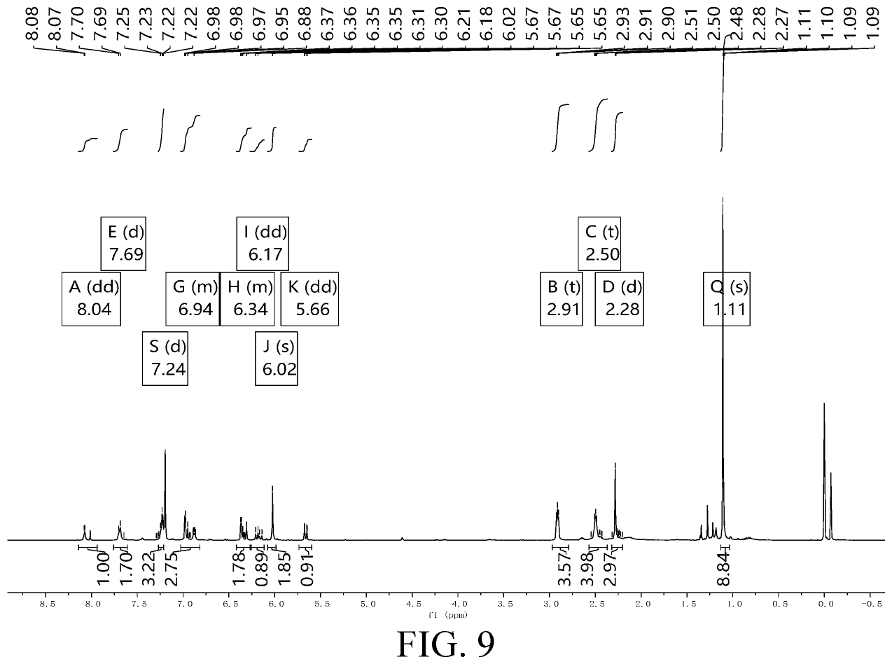

Synthesis of compound 7: To a round-bottom flask, were added t-BuOH (100 ml), compound 36 (4.7 g, 11 mmol), and 4-(4-methylpiperazine)-3-nitroaniline (18) (2.36 g, 10 mmol). The above reaction mixture was stirred 5-10 min at a speed of 360 rpm, to which were successively added K₂CO₃ (6.08 g, 44 mmol), Pd₂(dba)₃ (500 mg, 0.55 mmol), and XPhos (262 mg, 0.55 mmol). The reaction solution was stirred and reacted for about 6 h under refluxing at 110° C. TLC detection indicated that the reaction was completed (developing solvent: CH₂Cl₂/CH₃OH=100/10, v/v). About 3.5 g of the product (compound 7) was obtained, with a yield of about 56%. ¹H NMR spectrum of compound 7 is shown in FIG. 9. HRMS (ESI⁺) m/z: calcd for C₃₂H₃₆N₈O₆: 629.2758 [M+H]⁺, 651.2656 [M+Na]⁺; Found 629.2837 [M+H]⁺, 651.2679 [M+Na]⁺. ¹H NMR (400 MHz, Chloroform-d) δ 8.04 (dd, J=24.9, 2.0 Hz, 1H), 7.69 (d, J=6.6 Hz, 2H), 7.24 (d, J=6.6 Hz, 3H), 7.03-6.82 (m, 3H), 6.42-6.26 (m, 2H), 6.17 (dd, J=16.8, 10.2 Hz, 1H), 6.02 (s, 2H), 5.66 (dd, J=10.1, 1.4 Hz, 1H), 2.91 (t, J=4.7 Hz, 4H), 2.50 (t, J=4.7 Hz, 4H), 2.28 (d, J=3.6 Hz, 3H), 1.11 (s, 9H).

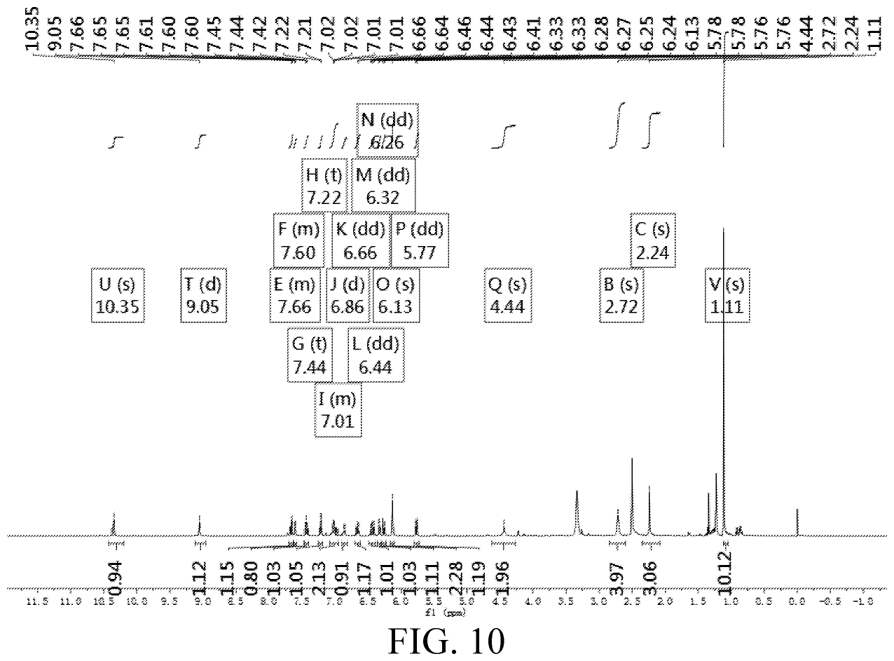

Synthesis of compound 8: compound 7 (628 mg, 1 mmol) was dissolved in the mixed solvent of ethanol and water, to which were added SnCl₂ (94.8 mg, 5 mmol) and 2 drops of concentrated hydrochloric acid. The reaction solution was heated under refluxing at 65° C. After reaction for 6 h under refluxing, TLC detection indicated that the reaction was completed (developing solvent: CH₂Cl₂/CH₃OH=5/1, v/v). The pH of the reaction solution was adjusted to be neutral with saturated Na₂CO₃ solution, and then the solution was extracted with CH₂C₁₂, to provide about 323 mg of the product as a brown solid (compound 8), with a yield of 54%. ¹H NMR spectrum of compound 8 is shown in FIG. 10. HRMS (ESI⁺) m/z: calcd for C₃₂H₃₈N₈O₄: 599.3016 [M+H]⁺; Found 599.3088 [M+H]⁺. ¹H NMR (600 MHz, DMSO-d₆) δ 10.35 (s, 1H), 9.05 (d, J=6.7 Hz, 1H), 7.69-7.63 (m, 1H), 7.62-7.58 (m, 1H), 7.44 (t, J=8.1 Hz, 1H), 7.22 (t, J=4.2 Hz, 1H), 7.07-6.95 (m, 2H), 6.86 (d, J=8.6 Hz, 1H), 6.66 (dd, J=18.1, 8.4 Hz, 1H), 6.44 (dd, J=17.0, 10.2 Hz, 1H), 6.32 (dd, J=11.9, 3.7 Hz, 1H), 6.26 (dd, J=16.9, 1.9 Hz, 1H), 6.13 (s, 2H), 5.77 (dd, J=10.2, 2.0 Hz, 1H), 4.44 (s, 2H), 2.72 (s, 4H), 2.24 (s, 3H), 1.11 (s, 9H).

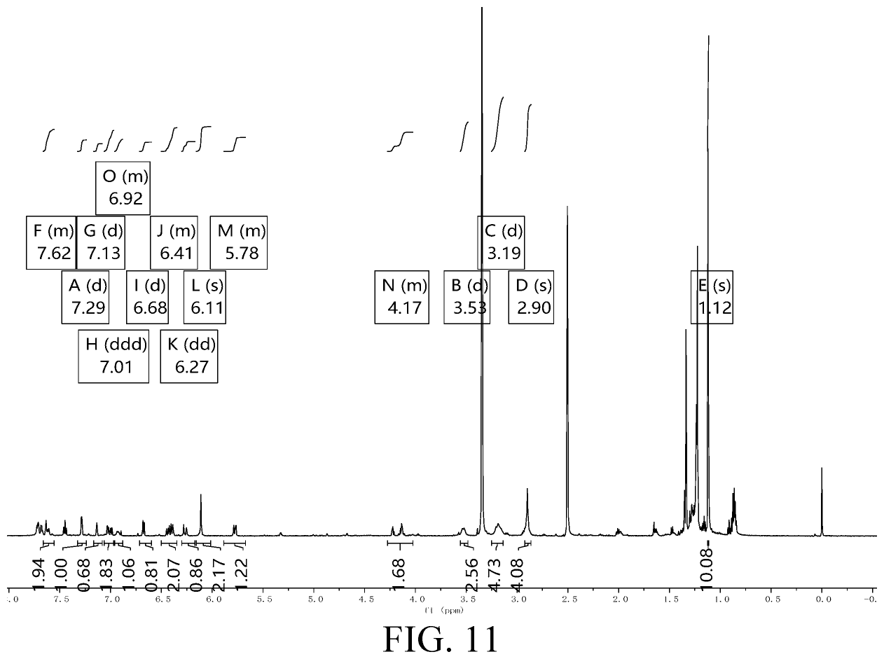

Synthesis of compound 9: compound 8 (180 mg, 0.3 mmol) was dissolved in a dilute HCl solution (125 μl was dissolved in 100 ml of water, and 50 ml was taken out), and then placed at −3° C. (in a low-temperature reactor); the solution of sodium nitrite (21 mg, 0.3 mmol) (dissolved in 5 ml of water) was added dropwise to the above mixed solution, and the mixture was stirred to diazotize. The temperature of the solution was kept at 0-5° C., but didn't rise to above 10° C. KI (250 mg, 1.5 mmol) was dissolved in 5 ml of water, and then added in the above solution. The resultant solution was stirred, and allowed to react overnight at room temperature. After completion of the reaction, the pH of the solution was adjusted to be neutral and in alkaline side with saturated Na₂CO₃ solution. The reaction solution was rotatory evaporated to dry, to which was added CH₂C₁₂ and water for extraction, followed by separation by column chromatography, to provide about 60 mg of the product (compound 9), with a yield of 28.2%. ¹H NMR spectrum of compound 9 is shown in FIG. 11. ¹H NMR (600 MHz, DMSO-d₆) δ 7.66-7.56 (m, 2H), 7.29 (d, J=3.6 Hz, 1H), 7.13 (d, J=2.5 Hz, 1H), 7.01 (ddd, J=20.5, 8.0, 2.4 Hz, 2H), 6.96-6.88 (m, 1H), 6.68 (d, J=8.3 Hz, 1H), 6.50-6.35 (m, 2H), 6.27 (dd, J=17.0, 1.9 Hz, 1H), 6.11 (s, 2H), 5.89-5.67 (m, 1H), 4.27-4.02 (m, 2H), 3.53 (d, J=11.3 Hz, 3H), 3.19 (d, J=14.4 Hz, 4H), 2.90 (s, 4H), 1.12 (s, 9H).

In the following, the beneficial effects of the present invention were demonstrated by specific experimental examples.

### Experimental Example 1. Anticancer Activity of the Compounds According to the Present Invention

Some materials and reagents mentioned in this experimental example are shown in Table 2, and the main instruments are shown in Table 3.

TABLE 2
Some experimental materials and reagents.

| Reagents | Manufacturer |
|---|---|
| Cell Counting Kit-8 (CCK8) | Sigma |
| BEAS-2B cells | BNCC |
| HBE cells | BNCC |
| H460 cells | Cell bank of Chinese |
| | Academy of Sciences |
| A549 cells | Cell bank of Chinese |
| | Academy of Sciences |
| H1975 cells | Cell bank of Chinese |
| | Academy of Sciences |
| HCC827 cells | Cell bank of Chinese |
| | Academy of Sciences |
| Fetal bovine serum | Gibco, USA |
| Annexin V-FITC apoptosis | Beijing 4A Biotech Co., Ltd |
| detection kit | |
| DMSO | Sigma |
| 96-well cell culture plate | Costar |
| 24-well cell culture plate | Costar |
| Trypsin | Gibco, USA |
| SDS-PAEG protein loading | Beyotime Biotechnology |
| buffer (5×) | |
| DMEM high-sugar medium | Gibco, USA |
| RPMI 1640 medium | Gibco, USA |
| Penicillin-streptomycin double | Gibco, USA |
| antibody | |
| 6-well cell culture plate | Corning, USA |
| PBS buffer | Gibco, USA |
| ERK½ Rabbit Polyclonal antidoby | proteintech |
| AKT Rabbit Polyclonal antibody | proteintech |
| p-ERK½ (T202/Y204) antibody | Cell Signaling Technology |
| p-EGFR (Ty992) antibody | Cell Signaling Technology |
| p-AKT (Ser473) antibody | Cell Signaling Technology |
| EGFR antibody | Cell Signaling Technology |
| Cell lysates | Beyotime Biotechnology |
| Tris-base | Abbexa |
| EDTA | Sigma |
| HRP-labeled goat anti-rabbit | Beijing Zhongshan Jinqiao |
| antibody | Biotechnology Co., Ltd |
| Glycine | Gibco, USA |
| NaCl | Chron Chmicals |
| SDS | Chron Chmicals |
| Tween-20 | Chengdu BEDIS Biotechnology |
| | Co., Ltd |
| Hanks solution | Corning, USA |

TABLE 3
Main instruments.

| Instruments | Model | Manufacturer |
|---|---|---|
| CO₂ incubator | Thermo311 | Thermo |
| Fluorescence inverted | TE2000-U | Nikon |
| microscope | | |
| Ultrapure water | HYRO 4.0 | ULUPURE |
| manufacturing system | | |
| Flow cytometer | FACS Valibur | BD company |
| Benchtop refrigerated | 22R | Beckman company |
| microcentrifuge | | |
| Automatic scanning | Multiskan Sky | Thermo company |
| multimode microplate | | |
| reader | | |
| Clean bench | SW-CJ-1F | Jiangsu Sujing Group |
| | | Co., Ltd |
| UV spectrophotometer | DU460 | Beckman |
| High-speed | D37520 | Heraeus |
| low-temperature | | |
| centrifuge | | |
| 4° C./−20° C. refrigerator | HXC-158 | Haier |
| Autoclave | MLS-3780 | Sanyo |
| Electronic balance | ALC-10.4 | Shanghai Precision |
| | | Scientific Instrument |
| | | Co., Ltd |
| Single channel adjustable | DS14833 | Thermo company |
| pipette | | |
| −80° C. ultra low | Forma | Thermo company |
| temperature refrigerator | | |
| Cell viability analyzer | Vi-Cell | Beckman |
| Biological safety cabinet | NSF49 | Thermo company |
| Constant temperature | SPH-211B-GZ | Shanghai Shiping |
| incubator shaker | | Experimental Equipment |
| | | Co., Ltd |
| Ultrasonic cell disruptor | JYD-650 | Shanghai Zhisun |
| | | Equipment Co. Ltd. |
| Liquid nitrogen tank | YDS-10-125F | Bender |
| Automatic gel imaging | GelDoc | Bio-Rad company, USA |
| system | | |
| Circulating thermostatic | ZSXH-618/625 | Shanghai Zhicheng |
| waterbath | | Analytical Instrument |
| | | Manufacturing Co., Ltd |

### 1. Experimental Methods

### 1.1 Preparation of Compound Solution

Compounds 1-9 of the present invention were respectively weighed and dissolved in 1 ml of dimethyl sulfoxide (DMSO), to prepare a 10 mM drug solution, which was stored in a refrigerator at −20° C. in the dark after packaging. The stock solution was diluted with the corresponding culture medium to the required concentration just before the experiment.

### 1.2 Cell Culture Experiment

HBE cells and BEAS-2B cells are normal lung bronchial epithelial cell lines, and both cells were cultured in DMEM high glucose medium (supplemented with 10% fetal bovine serum and 1% double antibody) to the logarithmic growth phase; H460 cells, A549 cells, H1975 cells and HCC827 cells are lung cancer cell lines, and all of them were cultured in RPMI1640 medium (supplemented with 10% fetal bovine serum and 1% double antibody) to the logarithmic growth phase. Among them, H1975 cells and HCC827 cells are EGFR mutant cells.

### 1.3 Inhibitory Experiment of Cell Proliferation (Cell Counting Kit-8, CCK8 Method)

When the cells were cultured to 90% of the growth density, the cells were digested with 0.25% trypsin to prepare a single cell suspension. The cells were counted, and then seeded in a 96-well plate at a density of 5000 cells/well, 100 μl for each well. The culture plate was moved into a CO₂ incubator and incubated for 24 h at 37° C. and 5% CO₂. Compounds at different concentrations were added to each well, 5 replicate wells for each concentration, and the cells were treated with the compounds for 48 hours. CCK8 detection reagent was added to each well according to the operation manual, and then the plate was further incubated for 1 h before terminating the culture. 450 nm wavelength was chosen, and the absorbance value of each well was adjusted on the microplate ELISA analyzer. The result was recorded. Time was taken as the horizontal axis, and absorbance as the vertical axis to plot a curve. The half inhibitory concentration (IC₅₀) was calculated by Bliss method. Each experiment was repeated at least 3 times.

### 1.4 Apoptosis Experiment

Apoptosis was detected by Annexin V-FITC/PI double staining method. A variety of different cells in the logarithmic growth phase were inoculated evenly in a 6-well plate at a concentration of 1×10⁶ cells/well, 2 ml for each well, and then incubated overnight. Then, different compounds were added, and each compound was set different concentrations. After 18 h, the cells were collected with 0.25% trypsin without EDTA and washed twice with PBS solution. Then, the cells were stained by the apoptosis detection kit according to the instructions, and analyzed by flow cytometry.

### 1.5 Cell Cycle Experiment

A variety of different cells in the logarithmic growth phase were inoculated evenly in a 6-well plate at a concentration of 1×10⁶ cells/well, 2 ml for each well, and then incubated overnight. Then, different compounds were added, and each compound was set different concentrations. After 18 h, the cells were collected, resuspended in PBS, and centrifuged to remove the supernatant, to which was then added 0.5 ml of pre-cooled 75% absolute ethanol dropwise, followed by vortexing to mix the cells and standing overnight at 4° C. The solution was centrifuged at 1000 rpm/min for 4 min, and the supernatant was discarded. Propidium iodide staining solution was prepared by using 5.6 ml staining buffer, 210 μl propidium iodide staining solution (25×), and 56 μl RNaseA (2.5 mg/ml). 0.4 ml of propidium iodide staining solution was added to each tube to resuspend the cells, and then incubated for 30 min at room temperature in the dark. The cell suspension was gently pipetted to mix, filtered into a flow tube, and stored in the dark at 4° C. The cells were detected on the flow cytometer within 1 h.

### 1.6 Related Protein Monitoring

### 1.6.1 Extraction of Total Cell Protein

(1) Various cell lines in the logarithmic growth phase were evenly inoculated in a 6-well plate at a concentration of 3×10⁶ cells/well, respectively, and cultured for 24 h;

(2) The culture supernatant was removed, and then the cells were washed twice with PBS, and treated with serum-free medium for 1 hour;

(3) The culture supernatant was removed, and each compound at different concentrations was added, then the cells were treated for 2 h;

(4) The supernatant was removed, and the cells were washed twice with pre-cooled PBS, then the supernatant was discarded;

(5) 100 μl of cell lysate was added to each well, and cells were lysed on ice for 10 min. The cells were scraped with a cell scraper. The cell lysate was transferred to a pre-cooled 1.5 ml EP tube, sonicated, and centrifuged under 12000 rpm at 4° C. for 10 min. The supernatant was carefully aspirated and stored at −20° C.

### 1.6.2 Determination of Protein Concentration by BCA Method

(1) 0 μl, 1 μl, 2 μl, 4 μl, 8 μl, 12 μl, 16 μl, 20 μl of BSA standard (0.5 mg/ml) were successively added to a 96-well culture plate, and then the total volume was made up to 20 μl with pre-cooled PBS;

(2) The test sample was diluted 20 times, and added to a 96-well plate at 20 l/well;

(3) 200 μl of BCA working solution was added to each well, and the plate was placed in a incubator and cultured for 30 min, then cooled to room temperature;

(4) The absorbance of each well was detected at 562 nm with a microplate reader, and the absorbance of water was set as zero;

(5) A protein standard curve was drawn to calculate the protein concentration of the test sample.

### 1.6.3 SDS-PAGE Protein Electrophoresis

(1) A separation gel with a concentration of 12% was prepared (see Table 4);

(2) The separation gel was poured between the two glass plates under the conditions of avoiding air bubbles, and added to 1 cm from the bottom edge of the comb, then the double-distilled water was gently added for water sealing;

(3) A concentrated gel with a concentration of 5% was prepared (see Table 5);

(4) After filling the separation gel, the glass plats were allowed to stand at room temperature for 30 min. When the separation gel was completely condensed, the double-distilled water on the upper layer was slowly poured out, and the remaining double-distilled water was absorbed with a filter paper strip;

(5) The concentrated gel was quickly injected to the top of the glass plates, and a comb was inserted to prevent air bubbles, then the glass plates were allowed to stand for 30 min at room temperature for later use;

(6) To the aliquoted total cell protein, was added 5 μl of 5× loading buffer, and the protein was denatured in a water bath at 100° C. for 10 min, then the sample was centrifugated and loaded;

(7) 4 μl of pre-stained protein marker was added to the holes on both sides of the protein sample;

(8) The electrophoresis apparatus was turned on, and once the desired bands were separated, the electrophoresis could be stopped.

TABLE 4
Preparation of 12% separation gel (15 ml).

| Reagent name | 12% separation gel (ml) |
|---|---|
| 30% Acrylamide | 6.00 |
| 1.5M Tris-HCl (pH 8.8) | 3.80 |
| 10% SDS | 0.15 |
| TEMED | 0.006 |
| 10% AP | 0.15 |
| Double-distilled water | 4.90 |

TABLE 5
Preparation of 5% concentrated gel (4 ml).

| Reagent name | 5% separation gel (ml) |
|---|---|
| 30% Acrylamide | 0.67 |
| 1.0M Tris-HCl (pH 6.8) | 0.5 |
| 10% SDS | 0.04 |
| TEMED | 0.004 |
| 10% AP | 0.04 |
| Double-distilled water | 2.7 |

### 1.6.4 Transmembrane

(1) PVDF membrane with suitable size was soaked in methanol for about 30 s, and then transferred to the transfer buffer;

(2) The “sandwich” of sponge pad-filter paper-separation gel-PVDF filter membrane-sponge pad was made, and put into the transfer tank;

(3) The transfer buffer was poured into the tank, in which the cooling device was placed;

(4) Transfer was carried out for 100 min under the condition of a constant current of 300 mA. After completion of transmembrane, the PVDF membrane was taken out, as well as the front and back sides, together with the position of the reference protein with standard molecular weight, were marked.

### 1.6.5 Blocking, Primary Antibody Incubation, Secondary Antibody Incubation

(1) The successfully transferred membrane was placed in the 1× blocking solution prepared, and kept in the blocking solution at room temperature for about 1 h;

(2) EGFR protein and its downstream proteins were diluted with 1× primary antibody diluent, and incubated overnight at 4° C., using 3-actin antibody as an internal reference;

(3) The membrane was washed 3 times with 1×TBST, 5 min for each time, and then washed with 1× secondary antibody dilution, followed by incubation for 1 h at room temperature. Finally, the membrane was washed 3 times with 1×TBST, 15 min for each time.

### 1.6.6 ECL Development

(1) ECL chemiluminescent solutions A and B were mixed in a ratio of 1:1, and the mixture was ready for use;

(2) The mixed ECL reagent was added to PVDF membrane (1 ml/10 cm²), and subjected to chemiluminescence to obtain a band;

(3) The pictures were taken using gel imaging system.

### 1.6.7 Analyzing the Influence of Each Compound on the Expression of Pathway Proteins by Western Blot Method

Western Blot method was used to analyze the influence of each compound on the expression of pathway proteins.

### 2. Experimental Results

### 2.1 Detecting the Inhibitory Effect of the Compound According to the Present Invention on the Proliferation of Different Lung Cancer Cell Lines by CCK8 Method

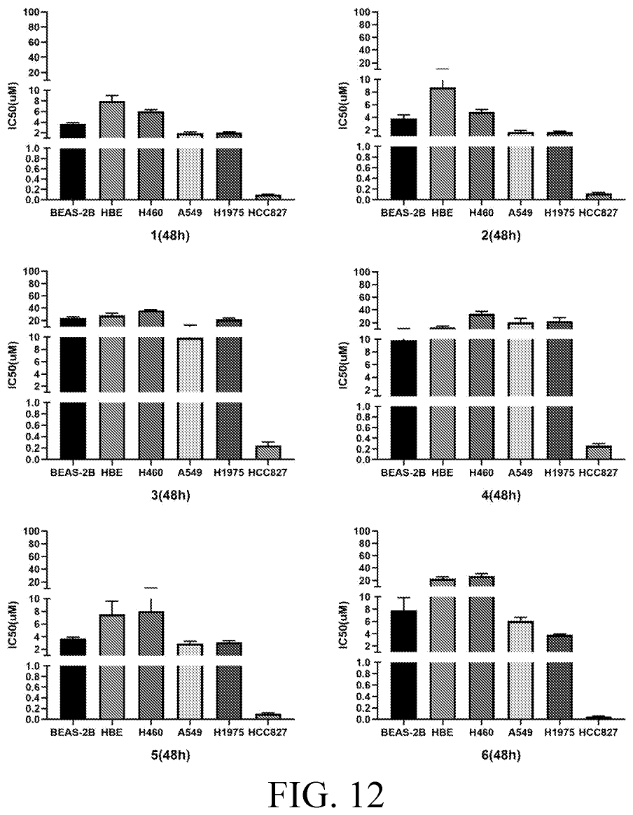

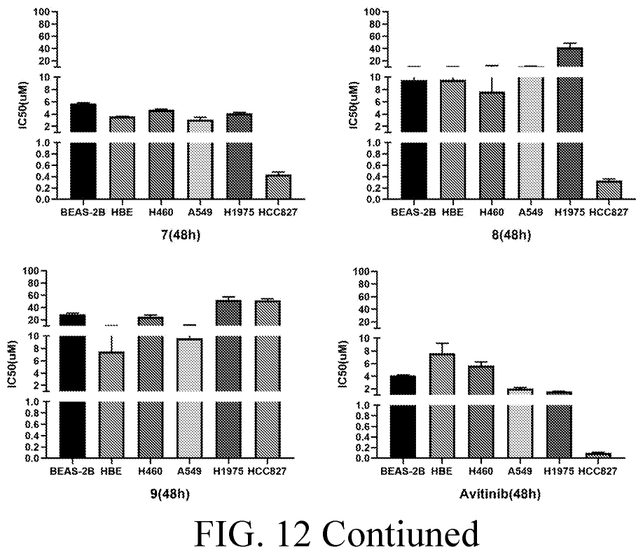

The inhibition of 9 target compounds according to the present invention on the proliferation of different lung cancer cells were tested at different concentrations (0 μmol, 3 μmol, 125 μmol, 6.25 μmol, 12.5 μmol, 25 μmol, 50 μmol) by CCK8 method, using commercially available avitinib as a positive control, and the activity against different lung cancer cells was evaluated. Normal lung cell lines and different lung cancer cell lines were selected for experiments. The results of the 48 h experiment are shown in Table 6 and FIG. 12. Amongst, the compound with high anti-cancer activity was screened, whose anti-tumor activity was further studied.

TABLE 6
Inhibitory activity of the compounds according to the present invention
against normal cells and different lung cancer cell lines (IC₅₀/μM).

| Compound | 1 | 2 | 3 | 4 | 5 | 6 | 7 | 8 | 9 | Avitinib |
|---|---|---|---|---|---|---|---|---|---|---|
| BEAS-2B | 3.68 | 3.80 | 23.80 | 9.89 | 3.68 | 7.80 | 5.73 | 9.54 | 29.11 | 4.11 |
| HBE | 8.00 | 8.73 | 28.06 | 12.50 | 7.54 | 22.66 | 3.61 | 9.58 | 7.50 | 7.62 |
| H460 | 6.02 | 4.86 | 36.02 | 33.58 | 8.02 | 26.67 | 4.68 | 7.65 | 25.12 | 5.68 |
| A549 | 1.92 | 1.68 | 9.92 | 20.63 | 2.90 | 6.06 | 3.10 | 10.75 | 9.63 | 2.01 |
| H1975 | 2.06 | 1.67 | 21.98 | 22.43 | 3.10 | 3.84 | 4.12 | 41.98 | 52.33 | 1.56 |
| HCC827 | 0.10 | 0.12 | 0.24 | 0.26 | 0.10 | 0.046 | 0.44 | 0.33 | 51.68 | 0.11 |

According to the experimental results, except for compound 9, all other compounds had certain inhibitory activity on the proliferation of tumor cell lines in vitro.

(1) Compared with the positive control commercially available Avtinib, the inhibitory activity of compound 1 (Avtinib synthesized in our lab) on human normal bronchial epithelial cells BEAS-2B cells and HBE cells, as well as on different lung cancer cells (human large cell lung cancer cells H460 cells, human lung adenocarcinoma cell A549 cells, EGFR mutant cells H1975 cells and HCC827 cells) was basically the same as that of the positive control.

(2) Compared with compound 1, the inhibitory activity of compound 2 on human normal bronchial epithelial cells BEAS-2B cells and HBE cells, as well as on different lung cancer cells (human large cell lung cancer cells H460 cells, human lung adenocarcinoma cells A549 cells, EGFR mutant cells H1975 cells and HCC827 cells) was basically the same as that of the positive control.

(3) Compared with compound 1, the inhibitory activity of compound 3 on human normal bronchial epithelial cells BEAS-2B cells and HBE cells, as well as on different lung cancer cells (human large cell lung cancer cells H460 cells, human lung adenocarcinoma cells A549 cells and EGFR mutant cells H1975 cells) was lower, but the inhibitory activity on HCC827 cells was similar to that of compound 1, indicating that compound 3 has good selectivity for HCC827 cells.

(4) The inhibitory effect of compound 4 on different cell lines was similar to that of compound 3.

(5) The inhibitory effect of compound 5 on different cell lines was similar to that of compound 2.

(6) The inhibitory effect of compound 6 on different cell lines was similar to that of compound 3.

(7) The inhibitory effect of compound 7 on different cell lines was similar to that of compound 2.

(8) Compound 8 has a certain inhibitory effect on HCC827 cells, but has relatively poor inhibitory effects on other cell lines.

(9) The inhibitory effect of compound 9 on different cell lines was poorer than that of other compounds. In summary, for different cell lines, compounds 2, 5, and 7 showed similar inhibitory effects to Avtinib, while compounds 3, 4, and 6 had high targeting selectivity for EGFR mutant HCC827 cells. In particular, compared with the positive control commercially available Avtinib, compound 6 is less toxic to normal epithelial cells HBE cells, and the selectivity coefficient exceeds 490 times (compared with the IC₅₀ values against HBE cells and HCC827 cells).

### 2.2 the Inhibition of Compound on EGFR Phosphorylation

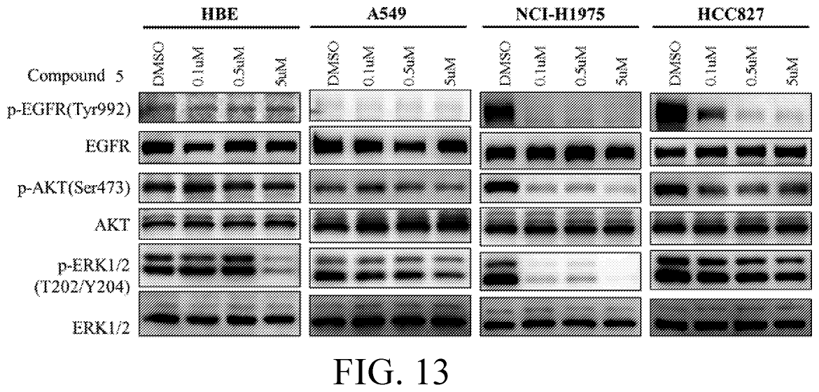

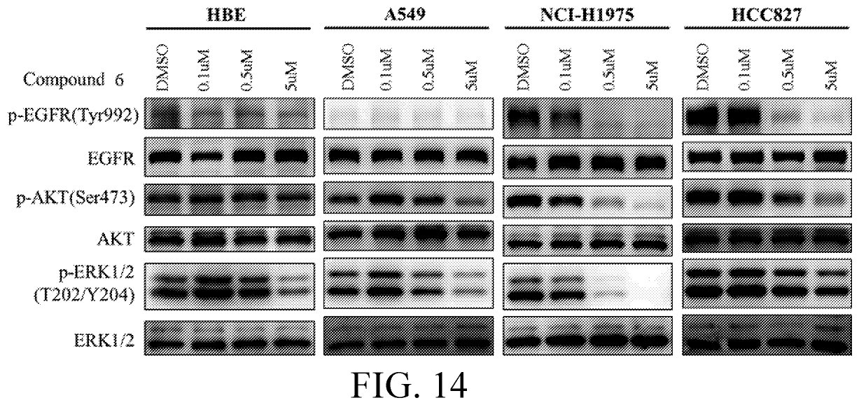

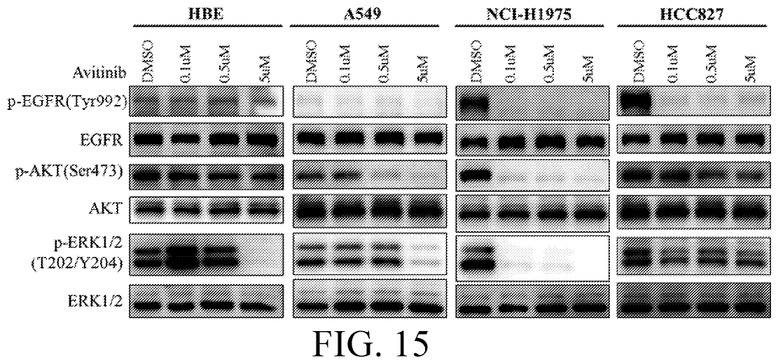

Western blot analysis confirmed that compounds 5 and 6 of the present invention effectively inhibited the phosphorylation of EGFR-Tyr992 in H1975 cells. In addition to inhibiting the phosphorylation of EGFR-Tyr992, both compounds also inhibited the phosphorylation of Akt and ERK1/2, two important downstream targets involved in cancer cell proliferation and survival in H1975 cells (FIG. 13 and FIG. 14). Consistent with the data of EGFR phosphorylation, both of two compounds had much weaker inhibitory effects on the phosphorylation of Akt and ERK1/2 in wild-type EGFR cells. In general, from the molecular mechanism, the test results of EGFR phosphorylation showed that the two newly synthesized compounds inhibited EGFR phosphorylation in the same way as Avitinib (FIG. 15), which inhibited EGFR T790M and other sensitive mutations.

### 2.3 Apoptosis Assay

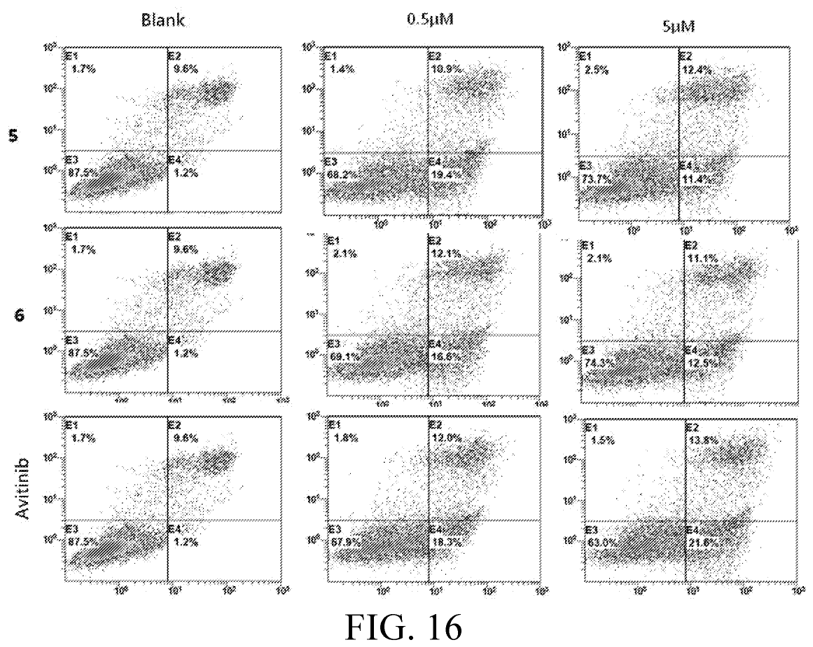

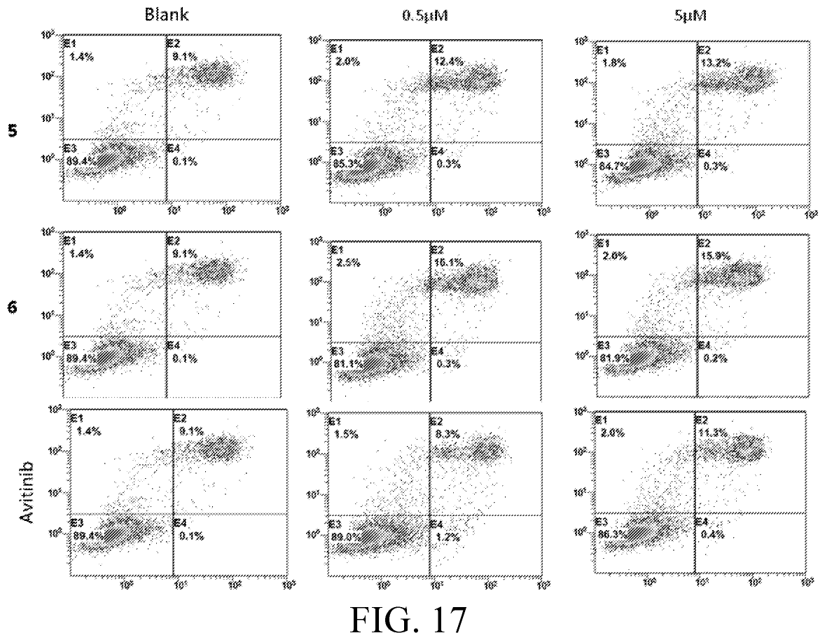

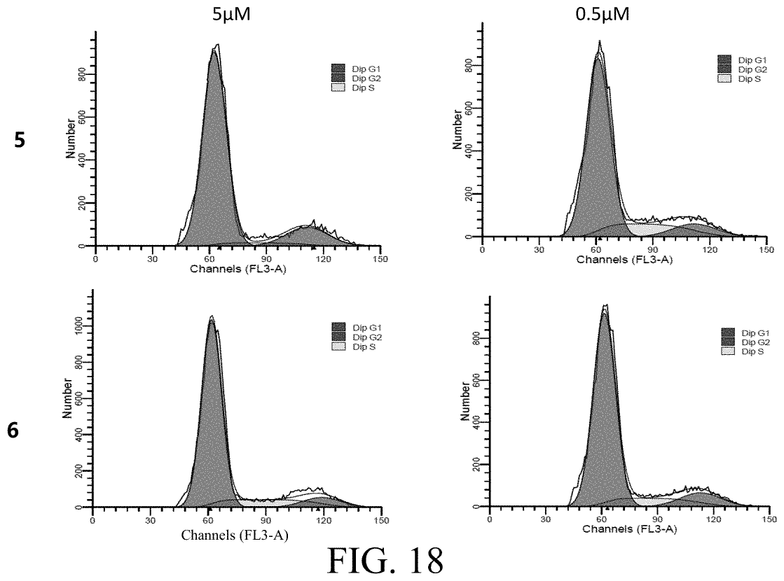

Due to the compound's good inhibitory effect on tumor cells, using compounds 5 and 6 as representatives, their apoptosis-inducing effects were further detected by flow cytometry. The results are shown in FIG. 16, FIG. 17, Table 7, and Table 8.

In the experiment, human lung cancer cells HCC827 cells and H1975 cells were selected as the detection system. The control group was a blank control (DMSO). The test concentrations of the compounds were 0.5 μM and 5 μM. After treatment with the compounds for 18 h, the flow cytometry was used for detection. In the apoptosis result graph, the cells in quadrant E1 are necrotic cells, the cells in quadrant E2 are late apoptotic cells, the cells in quadrant E3 area represent live cells, and the cells in quadrant E4 are early apoptotic cells. As shown in the experimental results, compounds 5 and 6 had certain apoptosis-inducing effects on HCC827 and H1975 cells.

TABLE 7
The effect of compound 5, compound 6 and Avtinib on the
apoptosis of HCC827 cells.

| | | Control | 0.5 μM | 5 μM |
|---|---|---|---|---|
| 5 | Total apoptosis rate | 10.8 | 30.3 | 23.8 |
| | (%) | | | |
| 6 | Total apoptosis rate | 10.8 | 28.7 | 23.6 |
| | (%) | | | |
| Avitinib | Total apoptosis rate | 10.8 | 30.3 | 35.4 |
| | (%) | | | |

TABLE 8
The effect of compound 5, compound 6 and Avtinib on the
apoptosis of H1975 cells.

| | | Control | 0.5 μM | 5 μM |
|---|---|---|---|---|
| 5 | Total apoptosis rate (%) | 9.2 | 12.7 | 13.5 |
| 6 | Total apoptosis rate (%) | 9.2 | 16.4 | 16.1 |
| Avitinib | Total apoptosis rate (%) | 9.2 | 9.5 | 11.7 |

### 2.4 Detection of Cell Cycle

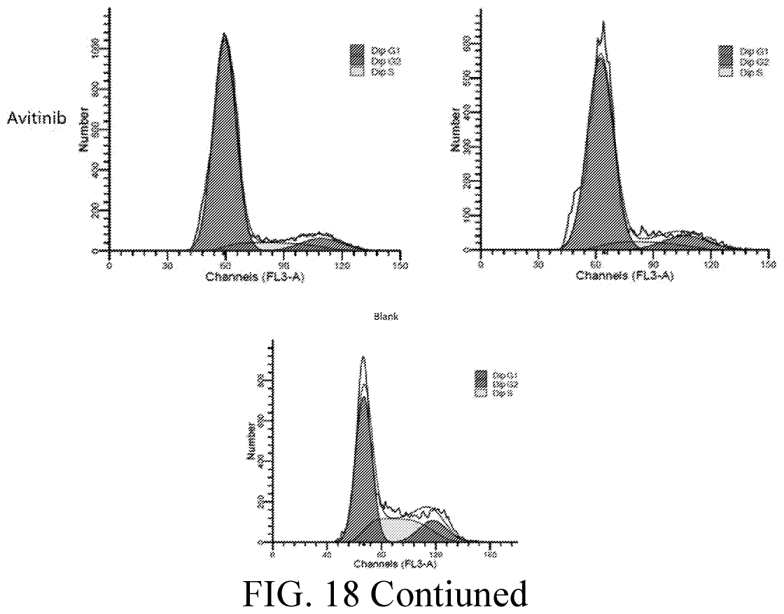

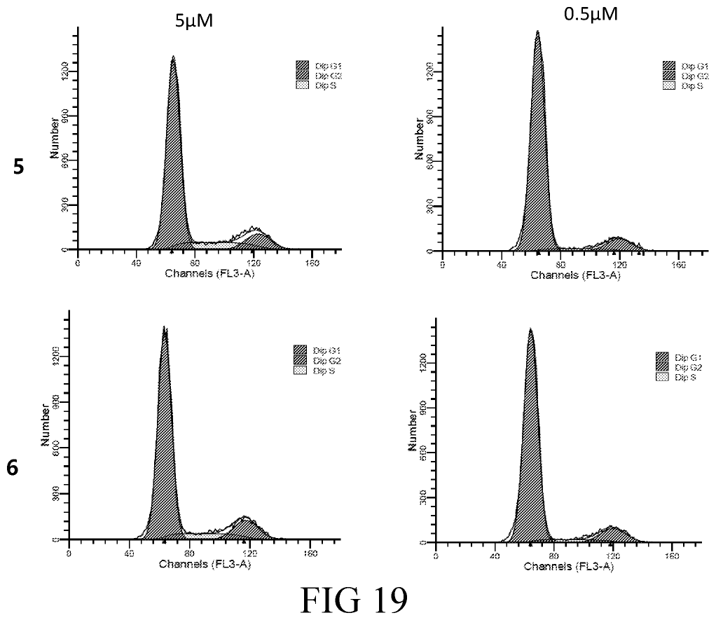

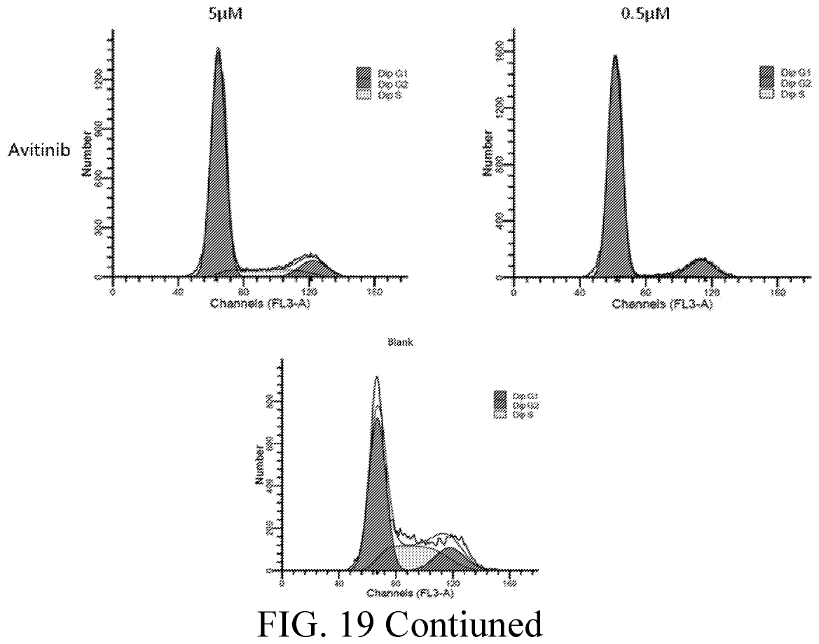

The cell cycle was detected by PI staining method, and the blocking effects of compounds 5 and 6 on HCC827 cells and H1975 cells were determined, which further proved that the drugs inhibited cell proliferation. The experimental results showed (FIG. 18, FIG. 19, Table 9, and Table 10) that compared with the control group, the ratio of two cells treated with the compounds in the G0/G1 phase was significantly higher. Correspondingly, the proportion of cells in S phase and G2/M phase decreased, indicating that the drug arrested the cell cycle of cancer cells in G0/G1 phase.

TABLE 9
The effects of compound 5, compound 6 and Avtinib on the cycle
of HCC827 cells.

| | | G0/G1 phase | S phase | G2/M phase |
|---|---|---|---|---|
| Compound | Concentration | (%) | (%) | (%) |
| 5 | Control | 41.35 | 33.66 | 24.99 |
| | 5 μM | 81.52 | 4.36 | 14.12 |
| | 0.5 μM | 73.29 | 16.99 | 9.72 |
| 6 | Control | 41.35 | 33.66 | 24.99 |
| | 5 μM | 78.12 | 13.93 | 7.95 |
| | 0.5 μM | 78.11 | 11.62 | 10.27 |
| Avitinib | Control | 41.35 | 33.66 | 24.99 |
| | 5 μM | 80.34 | 10.83 | 8.82 |
| | 0.5 μM | 80.57 | 9.09 | 10.34 |

TABLE 10
The effects of compound 5, compound 6 and Avtinib on the cycle
of H1975 cells.

| | | G0/G1 phase | S phase | G2/M phase |
|---|---|---|---|---|
| Compound | Concentration | (%) | (%) | (%) |
| 5 | Control | 55.23 | 30.25 | 14.52 |
| | 5 μM | 74.33 | 14.04 | 11.64 |
| | 0.5 μM | 86.20 | 4.64 | 9.17 |
| 6 | Control | 55.23 | 30.25 | 14.52 |
| | 5 μM | 76.23 | 10.59 | 13.18 |
| | 0.5 μM | 84.24 | 5.28 | 10.49 |
| Avitinib | Control | 55.23 | 30.25 | 14.52 |
| | 5 μM | 76.17 | 13.19 | 10.64 |
| | 0.5 μM | 83.79 | 3.74 | 12.47 |

### Experimental Example 2 Kinase Inhibitory Activity of the Compounds According to the Present Invention

According to relevant literature, the inhibitory activity of five kinases (EGFR, EGFR-LT, EGFR-LTC, BTK, JAK3) were tested. Note: EGFR, EGFR-LT, EGFR-LTC: wild-type EGFR, L858R/T790M (EGFR-LT), or L858R/T790M/C797S (EGFR-LTC) mutant protein. The inhibition rates of 10 compounds against different kinases at the same concentration (EGFR-80Nm, EFFR-LT-2 nM, EGFR-LTC-2 nM, BTK-4 nM, JAK3-1 nM) were tested, together with the IC₅₀ value of each compound (compounds 4, 5, and 6) against 5 different kinases.

Some materials and reagents described in this experimental example are shown in Table 11.

TABLE 11
Some experimental materials and reagents.

| Name | Manufacturer | Catalog No. | Batch No. |
|---|---|---|---|
| EGFR | Carna | 14-531 | WAB0528 |
| EGFR T790M L858R | Invitrogen | PR8911A | 1498821D |
| BTK | Carna | 08-180 | 14CBS-0619D |
| JAK3 | Carna | 08-046 | 13CBS-0637C |
| EGFR (T790M, | BPS | 40351 | 171103-G2 |
| C7975, L858R) | | | |
| Staurosporine | MCE | HY-15141 | 41248 |
| P2 | GL | 112394 | P131014-XP112394 |
| P22 | GL | 112393 | P180116-MJ112393 |
| Fluorescein-PolyGT | Invitrogen | PV3610 | 374293B |
| LanthaScreen ® | Invitrogen | PV3552 | 1508164B |
| Tb-PY20 | | | |
| Antibody Kit | | | |
| HEPES, pH7.5 | Gibco | 11344-041 | 1653630 |
| Brij-35 solution | Sigma | B4184 | 018K61251 |
| EDTA | Gibco | 15575-038 | 846901 |
| MgCl2 | Sigma | M2670-500g | BCBM7703V |
| DTT | Sigma | D0632-10G | SLBK4951V |
| DMSO | Sigma | D2650 | 474382 |
| EDTA | Sigma | E5134 | 60-00-4 |
| EGTA | Sigma | E3889-25G | 129K54001V |
| 96 well plate | Corning | 3365 | 22008026 |
| 384 well plate | Corning | 3573 | 35114024 |
| 384 well plate | Corning | 4512 | 13315017 |

### 1. Experiment of Mobility Shift Assay

### 1.1 Preparation of 1× Kinase Buffer and Stop Solution

### 1) 1× Kinase Buffer

50 mM HEPES, pH 7.5
0.0015% Brij-35

### 2) Stop Solution

100 mM HEPES, pH 7.5
0.015% Brij-35
0.2% Coating Reagent #3
50 mM EDTA

### 1.2 Preparation of Compound Solution

### 1) Dilution of Compound Solution

In the experiment on the IC₅₀ value of the compound against different kinases, the concentration was 10 μM, and the compound was formulated as a 50-fold concentration, that is, 500 μM. 95 μl of 100% DMSO was added to the first well of a 96-well plate, and then 5 μl of 10 mM compound solution was added to prepare a 500 μM compound solution. In the experiment on the single-concentration inhibition rate of the compound, each of the 10 compounds was set to different concentrations, corresponding to different kinases (EGFR-80 nM, EFFR-LT-2 nM, EGFR-LTC-2 nM, BTK-4 nM, JAK3-1 nM).

### 2) Transferring 5× Compound Solution to the Reaction Plate

10 μl solution was taken out from the 50-fold concentration of compound solution (prepared and tested), and transferred to a 96-well plate, to which was added 90 μl kinase buffer to prepare a 5-fold concentration of compound solution.

5 μl solution was taken out from the 5-fold concentration of compound solution in a 96-well plate, and transferred to a 384-well reaction plate. For example, the solution was transferred from well A1 of a 96-well plate to wells A1 and A2 of a 384-well plate, from well A2 of a 96-well plate to wells A3 and A4 of a 384-well plate, and so on.

### 1.3 Kinase Reaction and Termination

1) The kinase was added to 1× kinase buffer to form 2.5× kinase solution;

2) 10 μl of the above 2.5× kinase solution was transferred to a 384-well reaction plate, and 1× kinase buffer was added to the negative control well, then incubated for 10 min at room temperature;

3) FAM-labeled polypeptide and ATP were added to 1× kinase buffer to form a 2.5×substrate solution;

4) 10 μl of the above 2.5× substrate solution was transferred to a 384-well reaction plate;

5) The 384-well reaction plate was incubated at 28° C. for 60 min, to which was added 30 μl of stop solution to stop the reaction, and the model of biochemical incubator was SPX-100B-Z.

### 1.4 Data Reading

The conversion rate was read on CaliperEZ Reader.

### 1.5 Data Calculation

1) The conversion rate data were copied from CaliperEZ Reader II;

2) The conversion rate was converted into the inhibition rate;

Percent inhibition=(max−conversion)/(max−min)*100

“min” is the reading of the control well without enzyme; “max” is the reading of DMSO control well.

3) IC₅₀ values were fitted by using XLFit excel add-in version 5.4.0.8.

Fitting formula: Y=Bottom+(Top−Bottom)/(1+(IC50/X)\hat{ }HillSlope)

### 2. Lantha Screen Assay

### 2.1 Preparation of 1× Kinase Buffer

1× kinase buffer
50 mM HEPES, pH 7.5
0.0015% Brij-35

### 2.2 Preparation of Compound Solution

1) The initial detection concentration of the compound is 10 μM, and the compound was prepared into a 100-fold concentration, namely 1000 μM. 95 μl of 100% DMSO was added to the first well of a 96-well plate, and then 10 μl of 10 mM compound solution was added to prepare a 1000 μM compound solution. 50 μl of 100 μM compound solution prepared above was transferred to a 384-well Echo plate;

2) 50 μl of 100% DMSO was transferred to two empty wells as a control without compound and enzyme;

3) Echo 550 was used to transfer 100 nl of compound solution to a 384-well test plate. 2.3 Kinase reaction and termination

1) EGFR (T790M, C797S, L858R) was added to 1× kinase buffer to form 2× kinase solution;

2) 5 μl of the above 2× kinase solution was transferred to a 384-well reaction plate, and 1× kinase buffer was added to the negative control well, then incubated for 10 min at room temperature;

3) Fluorescein-PolyGT and ATP were added to 1× kinase buffer to form a 2× substrate solution;

4) 5 μl of the above 2× substrate solution was transferred to a 384-well reaction plate;

5) The 384-well reaction plate was incubated at room temperature for 30 min;

6) 2× mixed solution of antibody and EDTA was prepared, of which 10¹ was added to the 384-well reaction plate to stop the reaction;

7) The plate was left at room temperature for 60 min.

### 2.4 Data Reading

The excitation value at 340 nm and the emission values at 520 nm and 495 nm were read on Envision2014 Multilable Reader.

### 2.5 Data Calculation

1) The numerical ratio of the fluorescence reading (Lantha signal(520 nm/495 nm)) was copied; 2) The above data were converted into the inhibition percentage by the formula:

Percent inhibition=(max−Lantha signal)/(max−min)*100

“min” is the reading of the control well without enzyme; “max” is the reading of DMSO control well. 3) The data were imported into MS Excel, and IC50 results were subjected to curve fitting by using XLFit excel add-in version 5.4.0.8:

Fitting formula: Y=Bottom+(Top−Bottom)/(1+(IC₅₀/X)\hat{ }HillSlope)

### 3. Experimental Results

TABLE 12
Inhibitory activity of compounds 4, 5, and 6 on different
kinases (IC₅₀/nM).

| | | | | | EGFR |
|---|---|---|---|---|---|
| | | EGFR | | | (T790M, C797S, |
| Compound | EGFR | (T790M L858R) | BTK | JAK3 | L858R) |
| 4 | 217 | 3.1 | 31 | 2.3 | 1408 |
| 5 | 145 | 2.7 | 43 | 2.2 | 2225 |
| 6 | 64 | 3.4 | 11 | 2.2 | 464 |
| Staurosporine | 91 | 1.0 | 166 | 0.2 | 1.4 |

TABLE 13
Single-concentration inhibitory activity of a series of compounds
against EGFR (80 nM).

| | EGFR | | |
|---|---|---|---|
| | Compound | | |
| | Concentration | Mean | SD |
| Compound | (nM) | (100% inhibition) | (100% inhibition) |
| 1 | 80 | 80 | 2.5 |
| 2 | 80 | 86 | 3.2 |
| 3 | 80 | 32 | 5.3 |
| 4 | 80 | 32 | 12 |
| 5 | 80 | 37 | 4.8 |
| 6 | 80 | 63 | 0.3 |
| 7 | 80 | 4.7 | 5.5 |
| 8 | 80 | 4.5 | 0.6 |
| 9 | 80 | 3.6 | 1.1 |
| Avitinib | 80 | 76 | 3.8 |

TABLE 14
Single-concentration inhibitory activity of a series of compounds
against EGFR T790M L858R (2 nM).

| | EGFR (T790M L858R) | | |
|---|---|---|---|
| | Compound | | |
| | Concentration | Mean | SD |
| Compound | (nM) | (100% inhibition) | (100% inhibition) |
| 1 | 2 | 82 | 6.1 |
| 2 | 2 | 92 | 4.0 |
| 3 | 2 | 17 | 3.2 |
| 4 | 2 | 42 | 5.1 |
| 5 | 2 | 49 | 7.0 |
| 6 | 2 | 36 | 2.4 |
| 7 | 2 | 3.5 | 0.4 |
| 8 | 2 | 2.4 | 0.4 |
| 9 | 2 | 0.1 | 0.4 |
| Avitinib | 2 | 74 | 8.0 |

TABLE 15
Single-concentration inhibitory activity of a series of compounds
against EGFR (T790M, C7975, L858R) (2 nM).

| | EGFR (T790M, C7975, L858R) | | |
|---|---|---|---|
| | Compound | | |
| | Concentration | Mean | SD |
| Compound | (nM) | (100% inhibition) | (100% inhibition) |
| 1 | 2 | 8.8 | 6.6 |
| 2 | 2 | 9.2 | 7.0 |
| 3 | 2 | 8.2 | 11 |
| 4 | 2 | 7.8 | 5.6 |
| 5 | 2 | 5.8 | 6.4 |
| 6 | 2 | 8.0 | 6.6 |
| 7 | 2 | 8.4 | 4.8 |
| 8 | 2 | 3.6 | 2.9 |
| 9 | 2 | 2.1 | 4.4 |
| 10 | 2 | −0.7 | 3.1 |

TABLE 16
Single-concentration inhibitory activity of a series of compounds
on BTK (4 nM).

| | BTK | | |
|---|---|---|---|
| | Compound | | |
| | Concentration | Mean | SD |
| Compound | (nM) | (100% inhibition) | (100% inhibition) |
| 1 | 4 | 42 | 5.9 |
| 2 | 4 | 37 | 0.2 |
| 3 | 4 | 14 | 6.5 |
| 4 | 4 | 15 | 4.8 |
| 5 | 4 | 3.8 | 6.1 |
| 6 | 4 | 27 | 5.2 |
| 7 | 4 | 5.9 | 5.0 |
| 8 | 4 | −2.9 | 3.7 |
| 9 | 4 | 3.3 | 2.8 |
| Avitinib | 4 | 26 | 3.7 |

TABLE 17
Single-concentration inhibitory activity of a series of compounds
on JAK3 (1 nM).

| | JAK3 | | |
|---|---|---|---|
| | Compound | | |
| | Concentration | Mean | SD |
| Compound | (nM) | (100% inhibition) | (100% inhibition) |
| 1 | 1 | 68 | 1.2 |
| 2 | 1 | 77 | 2.8 |
| 3 | 1 | 11 | 3.3 |
| 4 | 1 | 26 | 0.1 |
| 5 | 1 | 23 | 3.8 |
| 6 | 1 | 29 | 0.3 |
| 7 | 1 | −1.4 | 0.0 |
| 8 | 1 | −7.6 | 4.4 |
| 9 | 1 | −4.0 | 0.7 |
| Avitinib | 1 | 56 | 3.1 |

From the above results, it could be seen that the IC50 values of compounds 4, 5, and 6 against EGFR L858R/T790M double mutation were 3.1 nM, 2.7 nM, 3.4 nM, respectively; compared with wild-type EGFR (IC₅₀ values being 217 nM, 145 nM, 64 nM, respectively), the inhibitory activity was 70-fold, 54-fold, and 18-fold higher, respectively, indicating that this series of compounds had good inhibitory activity and selectivity against mutant EGFR.

In summary, the compound of the present invention had low toxicity to normal cells, but had obvious inhibitory effect on lung cancer cell lines, and especially had good selectivity and significant inhibitory effect for EGFR mutant HCC827 cells. The compound of the present invention could induce the apoptosis of EGFR mutant cells H1975 cells and HCC827 cells, and at the same time block the cycle of both cells in the G0/G1 phase. Meanwhile, the compound of the present invention could effectively inhibit the phosphorylation of EGFR, as well as the phosphorylation of two important kinases Akt and ERK1/2 in the downstream of the pathway involved in the proliferation and survival of cancer cells in H1975 cells. In addition, the compound of the present invention had good inhibitory activity and selectivity for mutant EGFR. The compound of the present invention could be used to prepare drugs for the treatment of lung cancer, especially non-small cell lung cancer, which had a strong inhibitory effect on EGFR-mutant lung cancer and was less toxic. The present invention could also be used to prepare tyrosine kinase inhibitors, especially the inhibitors of EGFR phosphorylation, and had good application prospects.

1. A compound of formula I, or a salt thereof, or a stereoisomer thereof: 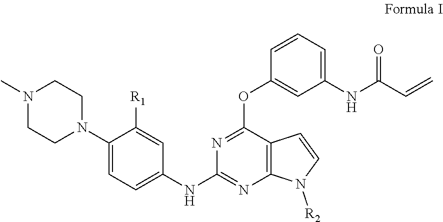
wherein,
R₁ is selected from the group consisting of halogen, C₁˜C₈ alkyl, C₁˜C₈ alkoxy, hydroxyl, nitro, amino or carboxyl; but R₁ is not a fluorine;
R₂ is selected from the group consisting of H, halogen, C₁˜C₈ alkyl, C₁˜C₈ alkoxy, hydroxyl, nitro, amino, carboxyl or —(CH₂)ₙ—O—C(O)—R₃;
n is selected from an integer from 1 to 8;
R₃ is selected from H or C₁˜C₈ alkyl.

2. The compound according to claim 1, or a salt thereof, or a stereoisomer thereof, characterized in that:
R₁ is selected from the group consisting of chlorine, bromine, iodine, C₁˜C₄ alkyl, C₁˜C₄ alkoxy, hydroxyl, nitro, amino or carboxyl;
R₂ is selected from the group consisting of H, halogen, C₁˜C₄ alkyl, C₁˜C₄ alkoxy, hydroxyl, nitro, amino, carboxyl or —(CH₂)ₙ—O—C(O)—R₃;
n is selected from an integer from 1 to 4;
R₃ is selected from H or C₁˜C₄ alkyl.
Preferably,
R₃ is selected from H or t-butyl.

3. The compound according to claim 1, or a salt thereof, or a stereoisomer thereof, characterized in that said compound has the structure of formula II. 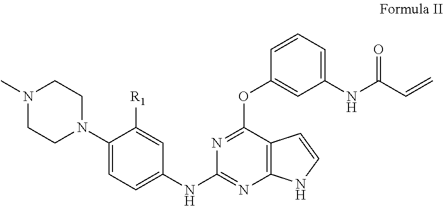
wherein,
R₁ is selected from the group consisting of halogen, C₁˜C₈ alkyl, C₁˜C₈ alkoxy, hydroxyl, nitro, amino or carboxyl; but R₁ is not a fluorine;
Preferably, R₁ is selected from the group consisting of chlorine, bromine, iodine, C₁˜C₄ alkyl, C₁˜C₄ alkoxy, hydroxyl, nitro, amino or carboxyl;
More preferably, R₁ is selected from the group consisting of chlorine, bromine, iodine, C₁˜C₃ alkyl, C₁˜C₃ alkoxy, nitro or amino.

4. The compound according to claim 1, or a salt thereof, or a stereoisomer thereof, characterized in that said compound has the structure of formula III: 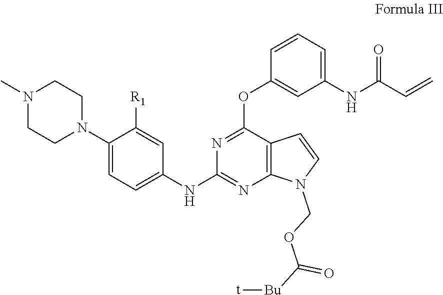
wherein,
R₁ is selected from the group consisting of halogen, C₁˜C₈ alkyl, C₁˜C₈ alkoxy, hydroxyl, nitro, amino or carboxyl; but R₁ is not a fluorine;
Preferably, R₁ is selected from the group consisting of chlorine, bromine, iodine, C₁˜C₄ alkyl, C₁˜C₄ alkoxy, hydroxyl, nitro, amino or carboxyl;
More preferably, R₁ is selected from the group consisting of chlorine, bromine, iodine, C₁˜C₃ alkyl, C₁˜C₃ alkoxy, nitro or amino.

5. The compound according to claim 1, or a salt thereof, or a stereoisomer thereof, characterized in that said compound is one of the following compounds.

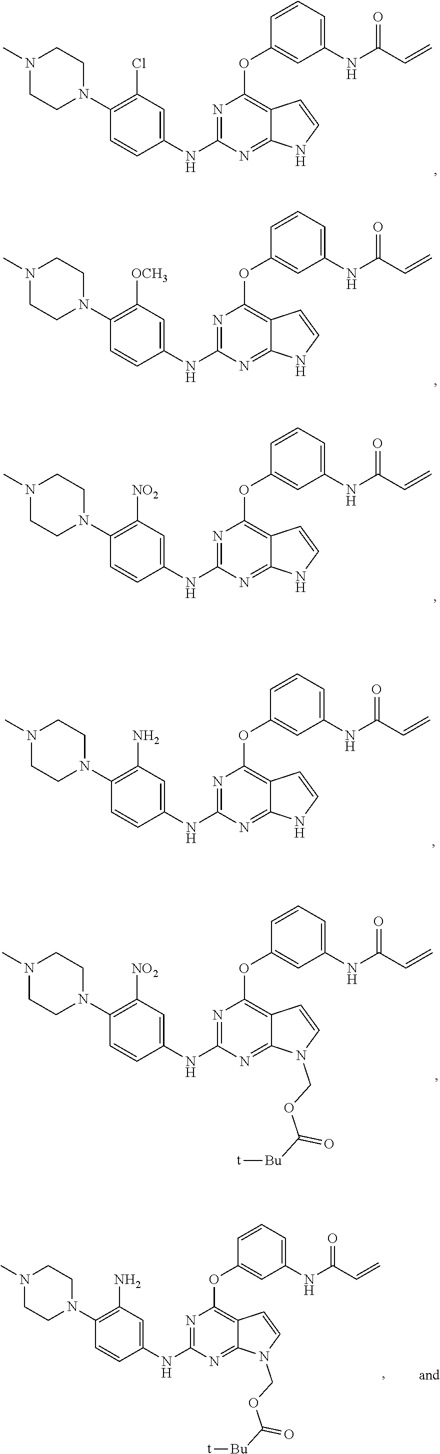

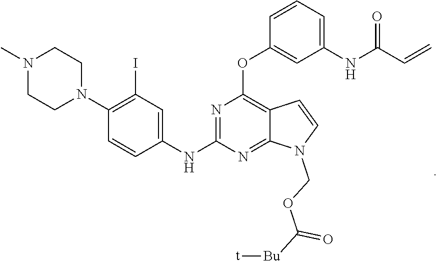

6. The use of the compound according to claim 1, or a salt thereof, or a stereoisomer thereof in the preparation of a tyrosine kinase inhibitor.

7. The use according to claim 6, characterized in that said tyrosine kinase inhibitor is a drug that inhibits the phosphorylation of EGFR.

8. The use according to claim 6, characterized in that said tyrosine kinase inhibitor is a drug for the treatment of cancers;
Preferably, the cancer is lung cancer, liver cancer, gastric cancer, kidney cancer, breast cancer, esophageal cancer, nasopharyngeal cancer, uterine cancer, colon cancer, rectal cancer, leukemia, bone cancer, and lymphoma.

9. The use according to claim 8, characterized in that the cancer is lung cancer; preferably, the lung cancer is non-small cell lung cancer; more preferably, the lung cancer is EGFR-mutant non-small cell lung cancer.

10. A drug, which is a preparation prepared from the compound according to claim 1, or a salt thereof, or a stereoisomer thereof as an active ingredient, with the addition of pharmaceutically acceptable excipients or auxiliary ingredients.
